(19) United States
(12) Patent Application Publication
(10) Pub. No.: US 2008/0039733 A1
(43) Pub. Date: Feb. 14, 2008

(54) SYSTEMS AND METHODS FOR CALIBRATION OF HEART SOUNDS

(76) Inventors: Kamil Unver, Santa Monica, CA (US); Damon J. Coffman, Portland, OR (US); Tat-Jin Teo, Sunnyvale, CA (US); Arvind Thiagarajan, Chennai (IN)

Correspondence Address: KANG LIM, 3494 CAMINO TASSAJARA ROAD #436, DANVILLE, CA, 94306, US

(21) Appl. No.: 11/762,930

(22) Filed: Jun. 14, 2007

Related U.S. Application Data
(60) Provisional application No. 60/821,752, filed on Aug. 8, 2006.

(51) Int. Cl. A61B 5/02

(52) U.S. Cl. 600/528; 600/300

(57) ABSTRACT

An auscultation system includes a transducer for generating an acoustic signal at a transducing location of the subject, and a sensor for receiving an attenuated acoustic signal at a sensing location of the subject. The attenuated signal received at the sensing location is digitized, and may be analyzed in the frequency and/or time domain. The comparison of the digitized attenuated signal against the initial transduced signal allows for the computation of the degree of acoustic attenuation between the transducing and sensing locations. Acoustic attenuation may be utilized to generate an intensity ratio. The ejection fraction of the heart subject may then be computed by correlation to the intensity ratio. Pulse echo methods are also disclosed. The echo transducer is oriented on the subject and generates a series of signal pulses. The return echo on the pulse is then received and a brightness encoded image is produced. The return echo provides location data on the internal structures of the subject including location, motion and speed.

### BACKGROUND OF THE INVENTION

This invention relates generally to medical electronic devices for analysis of auscultatory cardiac sounds. More particularly, this invention relates to a method for improving the analysis of heart sounds by compensating for acoustic attenuation of the human body, useful for example in measuring ejection fraction through auscultation.

The heart has four chambers—two upper chambers (called atria) and two lower chambers (ventricles). The heart has valves that temporarily close to permit blood flow in only one direction. The valves are located between the atria and ventricles, and between the ventricles and the major vessels from the heart. In healthy adults, there are two normal heart sounds: a first heart sound (S₁) and second heart sound (S₂). The first heart sound is produced by the closure of the Atrioventricular (AV) valves and the second heart sound is produced by semilunar valves closure. Because the heart is also divided into a “right side” and a “left side,” sometimes these sounds may be somewhat divided; most commonly noted is a “split S2,” caused when the right and left ventricles relax, and valves close at very slightly different times. Split S2 is normal, but occasionally the nature of the split can indicate an abnormality such as enlargement of one of the ventricles.

Moreover, in addition to these normal sounds a variety of other sounds may be present, including heart murmurs and adventitious sounds, or clicks. Murmurs are blowing, whooshing, or rasping sounds produced by turbulent blood flow through the heart valves or near the heart. Murmurs can happen when a valve does not close tightly, such as with mitral regurgitation which is the backflow of blood through the mitral valve, or when the blood is flowing through a narrowed opening or a stiff valve, such as with aortic stenosis. A murmur does not necessarily indicate a disease or disorder, and not all heart disorders cause murmurs.

Murmurs may be physiological (benign) or pathological (abnormal). Different murmurs are audible in different parts of the cardiac cycle, depending on the cause and grade of the murmur significant murmurs can be caused by: chronic or acute mitral regurgitation, aortic regurgitation, aortic stenosis, tricuspid stenosis, tricuspid regurgitation, pulmonary stenosis and pulmonary regurgitation

The first heart tone, or S₁, is caused by the sudden block of reverse blood flow due to closure of the mitral and tricuspid atrioventricular valves at the beginning of ventricular contraction, or systole.

The second heart tone, or S₂, marks the beginning of diastole, the heart's relaxation phase, when the ventricles fill with blood. The second heart sound is caused by the sudden block of reversing blood flow due to closure of the aortic valve and pulmonary valve. In children and teenagers, S2 may be more pronounced. Right ventricular ejection time is slightly longer than left ventricular ejection time.

A normal third heart sound or S₃ may be heard at the apex. This sound usually occurs approximately 0.15 seconds after the second heart sound. The third heart sound is a low pitched soft blowing sound. It may be caused by congestive heart failure, fluid overload, cardiomyopathy, or ventricular septal defect. The third heart sound usually occurs whenever there is a rapid heart rate, such as over 100 beats per minute (bpm). The third heart sound is caused by vibration of the ventricular walls, resulting from the first rapid filling. However, it may also be found in young persons, pregnant women or people with anemia with no underlying pathology.

The fourth heart sound S₄ occurs during the second phase of ventricular filling: when the atriums contract just before S1. As with S3, the fourth heart sound is thought to be caused by the vibration of valves, supporting structures, and the ventricular walls. An abnormal S4 is heard in people with conditions that increase resistance to ventricular filling, such as a weak left ventricle.

Auscultatory sounds have long been the primary inputs to aid in the noninvasive detection of various physiological conditions. For instance the stethoscope is the primary tool used by a clinician to monitor heart sounds to detect and diagnose the condition of a subject's heart. Auscultation itself is extremely limited by a number of factors. It is extremely subjective and largely depends on the clinician's expertise in listening to the heart sounds and is compounded by the fact that certain components of the heart sounds are beyond the gamut of the human ear.

In heart failure (HF) patients, the relative acoustic levels of the S₁ and S₂ heart sounds as heard by the stethoscope vary widely from that of a healthy person. In such HF patients the S₂ level is often greatly decreased, however, this problem is often not obvious due to the fact that the fat content and body variability change the acoustic levels, or due to the fact that the heart sounds are so faint the variation is difficult to discem. On the other hand pulmonary hypertension typically increases the P₂ component of S₂ level, but this also may be missed for the same reasons. What is desired is a way to calibrate, e.g., to normalize, the actual S₁ and S₂ levels at the locus or source inside the body of the HF patient when using a normal stethoscope chestpiece.

The first sound should be dependent on the effectiveness of the left heart. By collecting data, links may be established between the intensity of the first heart sound (S1) and the relative efficiency of the left heart. Specifically, ‘Ejection Fraction’ (EF), may be chosen as the measure of the effectiveness of the left heart. Ejection Fraction (EF) is the fraction of blood pumped out of a ventricle with each heart beat. The term ejection fraction applies to both the right and left ventricles; one can speak equally of the left ventricular ejection fraction (LVEF) and the right ventricular ejection fraction (RVEF). Without a qualifier, the term ejection fraction refers specifically to that of the left ventricle.

By definition, the volume of blood within a ventricle immediately before a contraction is known as the end-diastolic volume. Similarly, the volume of blood left in a ventricle at the end of contraction is end-systolic volume. The difference between end-diastolic and end-systolic volumes is the stroke volume, the volume of blood ejected with each beat. Ejection fraction (EF) is the fraction of the end-diastolic volume that is ejected with each beat; that is, it is stroke volume (SV) divided by end-diastolic volume (EDV). In a healthy 70-kg (154-lb) man, the SV is approximately 70 ml and the left ventricular EDV is 120 ml, giving an ejection fraction of 70/120, or 58%. Right ventricular volumes being roughly equal to those of the left ventricle, the ejection fraction of the right ventricle is normally equal to that of the left ventricle within narrow limits.

Damage to the muscle of the heart (myocardium), such as that sustained during myocardial infarction or in cardiomyopathy, impairs the heart's ability to eject blood and therefore reduces ejection fraction. This reduction in the ejection fraction can manifest itself clinically as heart failure. The ejection fraction is one of the most important predictors of prognosis; those with significantly reduced ejection fractions typically have a poorer prognoses.

Ejection fraction is currently commonly measured by echocardiography, in which the volumes of the heart's chambers are measured during the cardiac cycle. Ejection fraction can then be obtained by dividing stroke volume by end-diastolic volume as described above. Other methods of measuring ejection fraction include cardiac MRI, fast scan cardiac computed axial tomography (CT) imaging, ventriculography, Gated SPECT, and the MUGA scan. A MUGA scan involves the injection of a radioisotope into the blood and detecting its flow through the left ventricle. The historical gold standard for the measurement of ejection fraction is ventriculography. However, many of these methods are either expensive, uncomfortable, or require injection with radioactive material. Due to the cost benefits, ease of use, and minimally invasiveness of heart sound measurements, a preferred system of utilizing acoustic measurements to determine Ejection Fraction is desired.

Due to differences in the amount of soft tissue between different subjects, some way of calibrating data is required as to compare all subjects.

It is therefore apparent that an urgent need exists for an improved auscultatory device capable of calibrating the relative acoustic levels of heart sounds by compensating for the acoustic attenuation caused by the internal body structures and fluid boundaries of the patient; thereby, for example, enabling measuring of ejection fraction in a patient utilizing calibrated acoustic levels of heart sounds.

### SUMMARY OF THE INVENTION

To achieve the foregoing and in accordance with the present invention, a method and system of analyzing heart sounds is provided. Such an auscultation system is useful for a clinician to efficiently and accurately auscultate patients.

In one embodiment, the auscultation system includes a transducer for generating an acoustic signal at a transducing location of the patient, and a sensor for receiving an attenuated acoustic signal at a sensing location of the patient. The attenuated signal received at the sensing location is digitized, and may be analyzed in the frequency and/or time domain. The comparison of the digitized attenuated signal against the initial transduced signal allows for the computation of the degree of acoustic attenuation between the transducing and sensing locations.

The auscultation system, e.g., an electronic stethoscope, aids the clinician's diagnosis of the heart sounds by using the measured attenuation to calibrate the heart sounds received at the sensing location by a chest-piece of the auscultation system.

In some embodiments, the transducer is combined with an ECG sensor. In other embodiments, the transducer and the sensor are incorporated in a chestpiece for the auscultation system.

The method for measuring acoustic attenuation in a patient includes generating an acoustic signal on the patient and receiving an attenuated acoustic signal resulting from the original signal and computing an acoustic attenuation. The signal generation and sensing may be at separate locations or at a single location. In the event of a single location for both sensing and signal generation then a single transducer may be utilized to both generate acoustic signals and sense the resulting attenuation and heart signals.

When separate locations are utilized for signal generation and sensing the locations may be close together. The locations are located on a thoracic region of the patient. Additionally in some embodiments the sensing location may be a standard electrocardiogram position.

A noise canceller may be utilized in order to eliminate background noise during acoustic sensing. Then the heart sounds of the patient may be sensed and normalized based on the computed acoustic attenuation.

Also disclosed is a method for calibrating heart sounds of a subject, useful in association with an auscultation device having a transducer, a sensor and a heart sound processor. The transducer is oriented on the patient, as is the sensor. Sensing and transducing locations may be separate locations in close proximity.

An audio signal is generated by the transducer. The sensor receives the resulting attenuation signal and the heart signal. The heart signal may be filtered from the attenuation signal, and an acoustic attenuation may be computed. The heart sound may be calibrated from the computed acoustic attenuation.

Pulse echo methods are also disclosed, which is useful in association with an auscultation device having an echo transducer and a heart sound processor. The echo transducer is oriented on the patient and generates a series of signal pulses. The return echo on the pulse is then received and a brightness encoded image is produced. The return echo provides location data on the internal structures of the patient.

Heart sound signal of the patient are also received and filtered from the echo pulse. The heart sound is calibrated by acoustic properties of the tissues represented in the brightness encoded image.

Motion of the internal structures of the patient may be detected by comparing the subsequent brightness encoded images generated by subsequent pulses. Structure speed may also be determined by referencing the distance traveled by a time differential, wherein the time differential is computed by comparing times of generation of the different acoustic pulses. Additionally, analysis of Doppler shift between the generated acoustic pulse and the received echo may be utilized to determine speed of the moving internal structure.

Operating suggestions may be generated and displayed to the user by analyzing brightness encoded image for statistical confidence.

Additionally, a method for measuring the ejection fraction of a patient, useful in association with an auscultation device having a transducer, a sensor and a heart sound processor, is disclosed. The transducer and sensor are oriented on the patient. The transducer generates an acoustic signal, and the sensor receives the attenuated signal. The heart sounds are also received by the sensor and are filtered from the attenuation signal. The heart sound signals are then conditioned.

Acoustic attenuation may then be computed and utilized to generate an intensity ratio. The ejection fraction of the heart patient may then be computed by correlation to the computed intensity ratio.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

These and other features of the present invention will be described in more detail below in the detailed description of the invention and in conjunction with the following figures.

In order that the present invention may be more clearly ascertained, one or more embodiments will now be described, by way of example, with reference to the accompanying drawings, in which:

FIG. 1A illustrates an exemplary pair of transducing and sensing positions for measuring acoustic attenuation of a thoracic region in accordance with the present invention;

FIG. 1B illustrates an exemplary single location echo method for measuring acoustic attenuation of a thoracic region in accordance with the present invention;

FIG. 2 shows exemplary frontal ECG sensing positions located on the thoracic region;

FIG. 3A shows a front view illustrating one embodiment of a chest-patch which combines an ECG sensor with an acoustic transducer for the auscultation device of the present invention;

FIG. 3B shows a side view illustrating one embodiment of a chest-patch which combines an ECG sensor with an acoustic transducer for the auscultation device of the present invention;

FIG. 4A shows a front view illustrating another embodiment of a rectangular chest-patch which combines an ECG sensor with an acoustic transducer for the auscultation device of the present invention;

FIG. 4B shows a side view illustrating another embodiment of a rectangular chest-patch which combines an ECG sensor with an acoustic transducer for the auscultation device of the present invention;

FIG. 5 shows a side view illustrating one exemplary chest-piece which combine an acoustic transducer with an acoustic sensor for the auscultation device of the present invention;

FIG. 6 shows a side view illustrating a second exemplary chest-piece which combine an acoustic transducer with an acoustic sensor for the auscultation device of the present invention;

FIG. 7 shows a bottom view illustrating a third exemplary chest-piece which combines an acoustic transducer with an acoustic sensor in separate acoustic cavities for the auscultation device of the present invention;

FIG. 8A is a bottom view illustrating yet another chest-pad which includes a triplet of Acoustic Sensors in accordance with an embodiment of the present invention;

FIG. 8B is a bottom view illustrating yet another chest-pad which includes a quintuplet of Acoustic Sensors in accordance with an embodiment of the present invention;

FIG. 8C is a bottom view illustrating yet another chest-pad which includes a sextet of Acoustic Sensors in accordance with an embodiment of the present invention;

FIG. 9 shows an exemplary diagram of pressure, timing, blood volume and signals associated in a typical cardiac cycle;

FIG. 10A shows a functional block diagram of one embodiment of the auscultatory device in accordance with an embodiment of the present invention;

FIG. 10B shows a functional block diagram of another embodiment of the auscultatory device in accordance with an embodiment of the present invention;

FIG. 10C shows a functional block diagram of yet another embodiment of the auscultatory device in accordance with an embodiment of the present invention;

FIG. 10D shows a functional block diagram of yet another embodiment of the auscultatory device in accordance with an embodiment of the present invention;

FIG. 11 shows an illustration of a functional block diagram for the analyzer in accordance with an embodiment of the present invention;

FIG. 12 provides a detailed block diagram illustrating heart sound signal conditioner in accordance with an embodiment of the present invention;

FIG. 13 shows an exemplary process for self calibration of heart signals utilizing an embodiment of the auscultatory device;

FIG. 14 shows an exemplary process for signal conditioning of heart signals utilizing an embodiment of the auscultatory device;

FIG. 15 shows an exemplary process for generating the attenuation matrix utilizing an embodiment of the auscultatory device;

FIG. 16 shows an exemplary process for pulsed echo utilizing an embodiment of the auscultatory device;

FIG. 17 shows an exemplary process for motion detection in pulsed echo utilizing an embodiment of the auscultatory device;

FIG. 18 shows an exemplary process for structure speed detection in pulsed echo utilizing an embodiment of the auscultatory device;

FIG. 19 shows an exemplary process for using the auscultatory device to determine ejection fraction in accordance with an embodiment of the present invention;

FIG. 20 shows an exemplary process for signal processing for ejection fraction determination in accordance with an embodiment of the present invention;

FIG. 21A shows an exemplary illustration of ECG and sound wave measurements for usage by ejection fraction analysis;

FIG. 21B shows an exemplary illustration of sound wave measurements for usage by ejection fraction analysis;

FIG. 22 shows an exemplary illustration of isolated ECG and sound wave measurement for usage by ejection fraction analysis;

FIG. 23A shows an exemplary illustration of ECG and sound wave measurements when attenuation signal is applied for usage by ejection fraction analysis;

FIG. 23B shows an exemplary illustration of sound wave measurements when attenuation signal is applied for usage by ejection fraction analysis;

FIG. 24 shows an exemplary illustration of filtered measured transduction signals for ejection fraction analysis;

FIG. 25A shows an exemplary illustration of pre-filtered measured heart signals for ejection fraction analysis;

FIG. 25B shows an exemplary illustration of post-filtered measured heart signals for ejection fraction analysis; and

FIG. 26 shows an exemplary illustration of measured heart signals before and after de-noising for ejection fraction analysis.

### DETAILED DESCRIPTION OF THE INVENTION

The present invention will now be described in detail with reference to several embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention. The features and advantages of the present invention may be better understood with reference to the drawings and discussions that follow.

Systems and methods for heart auscultation are provided. The present invention utilizes active sound signals in order to generate attenuation of sound signals through patient. In some embodiments, a transducer provides an active acoustic signal. The signal may be continuous wave or pulse in nature. At least one sensor perceives the signal that has propagated through the patient's soft tissue. The sensor may, in some embodiments, be coupled to an electronic stethoscope or similar device. Such devices may include additional signal processing as is desired. The received signal may be analyzed for attenuation caused by structures in the patient. These measurements may be utilized to calibrate the perceived heart sounds.

Alternatively, in some embodiments, the generated signal may be a pulse in nature which reflects within the body causing an echo. The sensor may perceive the echo to generate a brightness representation of reflectivity due to impedance inhomogeneities in the body. Motion, location and speed of valve closure may additionally be determined.

Additionally, Ejection Fraction (EF) may be computed by correlation to a calibrated first (S1) heart sound and heart sound ratios. Electrocardiogram measurements and catheter readings may be utilized to confirm Ejection Fraction values.

The present invention will be disclosed as a series of electromechanical auscultation devices enabled to generate and perceive the required signals. Additional methods for the use of such devices for calibration, pulsed echo and ejection fraction will also be discussed.

Particular subheadings are included to provide guidance and organization to the disclosure. These sub headings are not intended to suggest or impose limitations upon the disclosed invention.

### Auscultation Devices

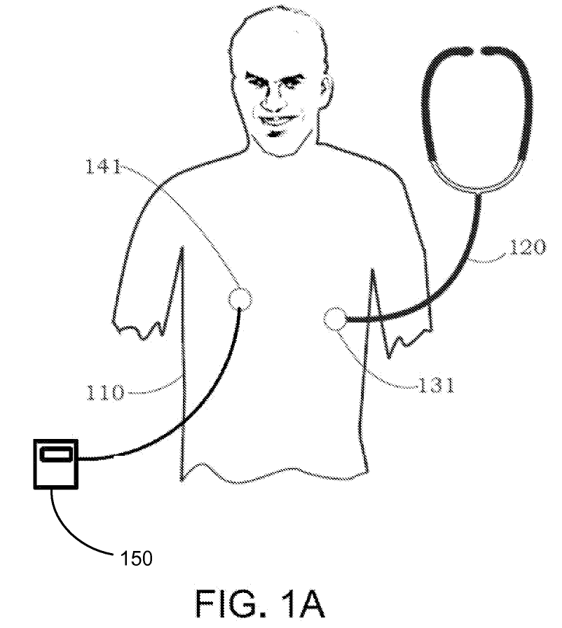

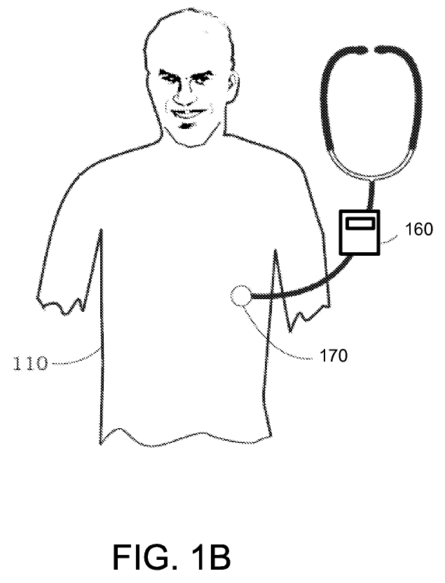

To facilitate discussion, FIG. 1A shows an exemplary pair of transducing and sensing positions for measuring acoustic attenuation of the thoracic region 110 using an auscultation device, e.g., an Electronic Stethoscope 120, of the present invention. Such an auscultation device includes an Acoustic Transducer 150 coupled to transducing position 141, and an acoustic sensor or stethoscope 120, coupled to sensing location 131. Additional pairs of transducing and sensing positions may be used to generate an acoustic attenuation map of thoracic region 110.

A suitable acoustic signal of known amplitude and frequency, e.g. a sine wave, may be provided by the Acoustic Transducer 150 at Transducing Location 141. Since one primary object of the invention is to measure and compensate for the acoustic attenuation of S1, S2, S3, S4 heart sounds and heart murmurs as these heart sounds travel from the heart to the acoustic sensor of Stethoscope 120, the acoustic signal may include a frequency range of about 50 Hz to 300 Hz. Depending on the implementation, this acoustic signal may include a series of stepped frequencies, a swept range of frequencies and/or multi-frequency signals.

In alternate embodiments, the acoustic signal from the transducer may have an acoustic frequency of 1 MHz and higher. Such embodiments enable the transducer signal to be filtered from the heart sounds by the Stethoscope 120. Additionally, such frequency range may provide directional information through Doppler analysis that would not be ascertainable at lower frequency transducer signal.

Additionally, in some embodiments the transducer signal may be pulsed as to minimize interference with the Stethoscope 120 microphone. Such a pulsed transducer signal, or echo pulse, may be relatively short, e.g. on the order of microseconds up to tens of microseconds.

The attenuated signal received at Sensing Location 131 is digitized, and may be analyzed in the frequency and/or time domain. For example, comparison of the digitized attenuated signal against the initial transduced signal allows for the computation of the degree of attenuation between Location 141 and Location 131. The computed degree of attenuation may be a single constant of volume attenuation or a multi-value measurement of attenuation of volume at one or more frequencies. This measurement of attenuation may also include time variant measurements as a function of frequency. Other standard signal processing techniques known to one skilled in the arts may also be used to compute attenuation.

By taking measurements from suitable pairs of transducing and sensing locations distributed over the area of interest, a matrix of the attenuation may be compiled. Subsequently, this attenuation matrix may be used to calibrate heart sounds to compensate for acoustic attenuation caused by the intervening tissues and fluids between the heart and the sensor, thereby increasing the accuracy of the diagnosis of the various heart sounds and murmurs.

FIG. 1B shows an exemplary diagram of transducer placement for pulse echo devices. In such embodiments the transducer and sensor may be located within the Echo Auscultation Devise 160. Thus, in these embodiments, the Sensing Location 131 and Transducing Position 141 may be adjacent to one another, or may be the same Common Location 170.

The Echo Auscultation Devise 160 provides the acoustic signal and subsequently senses the return echo, at the same Common Location 170 on the patient. Thus comfort and simplicity of the system is improved since there is only one pad needed.

As noted above, a suitable acoustic signal of known amplitude and frequency, e.g. a sine wave, may be provided by the acoustic transducer portion of the Echo Auscultation Device 160 at the Common Location 170. Again, the acoustic signal may include a frequency range of about 50 Hz to 300 Hz or may have an acoustic frequency of 1 MHz and higher. Depending on the implementation, this acoustic signal may include a series of stepped frequencies, a swept range of frequencies and/or multi-frequency signals.

Additionally, in some embodiments the transducer signal may be pulsed as to minimize interference from acoustic signal generation and acoustic measurements. Such a pulsed transducer signal, or echo pulse, may be relatively short, e.g. on the order of tens of microseconds.

The pulse echo is received at the Common Location 170, where it is digitized, and may be analyzed in the frequency and/or time domain. Other standard signal processing techniques known to one skilled in the arts may also be used to compute analysis. Echo patterns may be compiled within an attenuation matrix, which may be used to calibrate heart sounds to compensate for acoustic attenuation caused by the intervening tissues and fluids between the heart and the sensor, thereby increasing the accuracy of the diagnosis of the various heart sounds and murmurs.

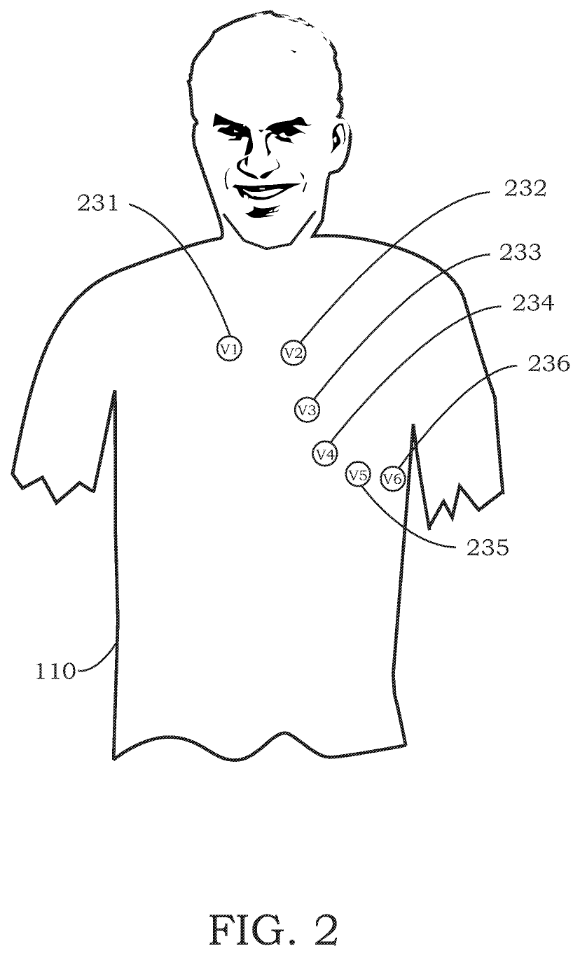

FIG. 2 shows a selection of suitable auscultation sensing locations. These locations include aortic, pulmonary, mitral, tricuspid and apex locations. Other exemplary sensing locations include typical ECG sensing locations 231, 232, 233, 234, 235, 236 corresponding to anterior thoracic ECG positions V1, V2, V3, V4, V5 and V6 may also be used as shown in FIG. 2. Additional thoracic ECG sensing locations such as posterior ECG positions V7, V8 and V9 (not shown) may also be used. Other auscultation locations known to one skilled in the cardiac diagnostic arts may also be used.

In some embodiments, the method for measuring heart sounds is performed to identify motion within the chest cavity. When the sensory location is fixed on the patient's torso, the received acoustic signals are processed for structures and fluids along the acoustic path.

A “brightness line” image may be generated from the received acoustic signals as to provide a representation for the structures along the acoustic path. By maintaining a fixed acoustic path, and repeatedly sensing the structures, motion may be identified and tracked. A heart valve is in motion with respect to the patient's chest wall, thus the distance of the valve to the chest wall may be deduced. Such a deduction may accurately be used to enable the calibration of the heart sound of that particular patient to his chest size or attenuation characteristics (the amount of subcutaneous fat, for example).

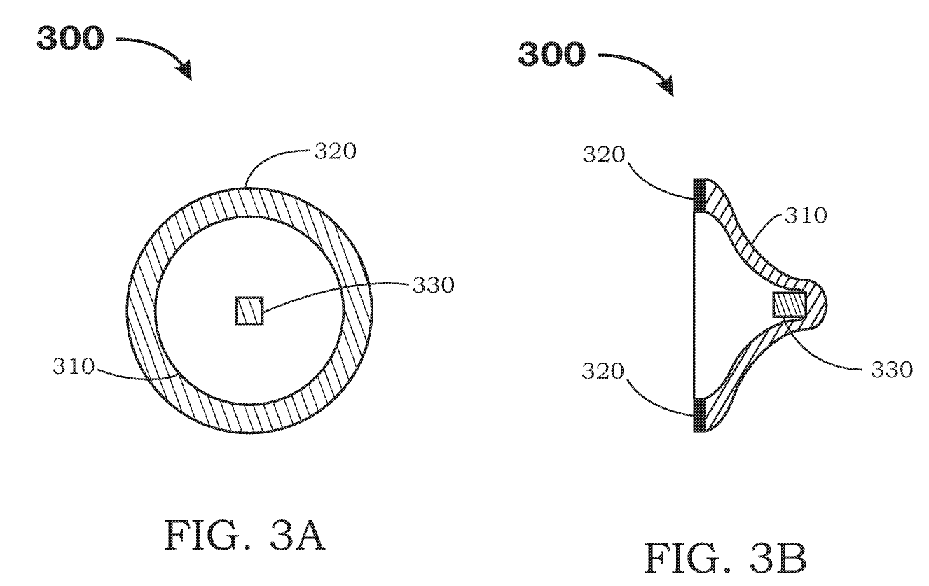

FIGS. 3A and 3B are front and side views illustrating one embodiment 300 of the present invention which combines an ECG sensor 320 and an acoustic transducer 330 housed in a bell-shaped body 310. In this embodiment, ECG sensor 320 is a conductive ring allowing ECG electrical signal transmission from the base of body 310. The bell-shaped body 310 focuses the acoustic signal generated by acoustic transducer 330, e.g., a miniature speaker, located at the top of body 310. ECG sensor 320 may include a sealing membrane to ensure both electrical conduction and mechanical air seal for superior acoustic transmission. Sealing may also be accomplished by an ECG gel in combination with or in place of a sealing membrane. Bell-shaped body 310 may be filled with air or fluid to facilitate acoustic transmission.

The Acoustic Transducer 330 may, in some embodiments, may be a traditional membrane and magnet speaker. Alternatively, Acoustic Transducer 330 may be a piezo transducer. Of course additional transducers may be utilized as is known by those skilled in the art.

A piezo Acoustic Transducer 330 may be capable of producing an acoustic signal, as well as sensing acoustic waves. Thus the Acoustic Transducer 330, in some embodiments where piezo or similar designs are utilized, may both supply the acoustic signal as well as provide sensory reception. Such a transducer may be utilized in the Pulse Echo Unit 170 of FIG. 1B. In these embodiments the Acoustic Transducer 330 provides a pulse of acoustic signal. During pulse generation the Acoustic Transducer 330 is unable to provide sensory, thus the length of pulse may be limited to a practical duration. In some embodiments, pulse duration of 10-30 microseconds is sufficient. The average cardiac cycle is on the magnitude of a full second, thus the pulse is a relatively short time for the Acoustic Transducer 330 to be unable to sense acoustic signals. Moreover, by interleaving the pulse and heart sounds over the cardiac signal, data loss may be mitigated.

In some alternate embodiments, the Acoustic Transducer 330 may be designed to only generate acoustic signals. Such an embodiment may be utilized in the separated Acoustic Transducer 150 and Stethoscope 120 design as illustrated in FIG. 1A. In these embodiments the Acoustic Transducer 330 may provide pulse acoustic signals, constant acoustic signals or a combination thereof.

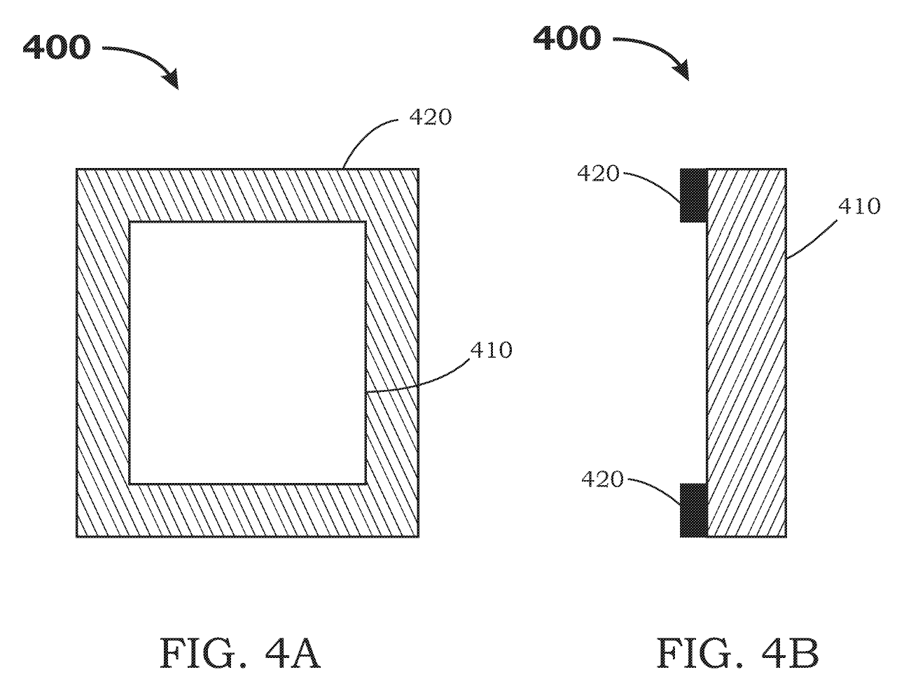

FIGS. 4A and 4B are front and side views illustrating one embodiment 400 of the present invention which combines an ECG sensor 420 and an acoustic transducer in a flat housing 410 which may be square-shaped as shown, or may be another suitable shape such as rectangular, polygonal, or oval. Acoustic transducer may be a piezoelectric element coupled to the base of housing 410, or may include additional acoustic generator designs, such as traditional speakers.

Again, the embodiment seen generally at 400 may include both acoustic generation and sensory, or may be limited to generation only, dependent on whether an echo type design, or a separated transducer and sensor design is required, as seen in FIG. 1B and 1A respectively.

ECG sensor 420 may include a sealing membrane to ensure both electrical conduction and mechanical air seal for superior acoustic transmission. Sealing may also be accomplished by an ECG gel in combination with or in place of a sealing membrane.

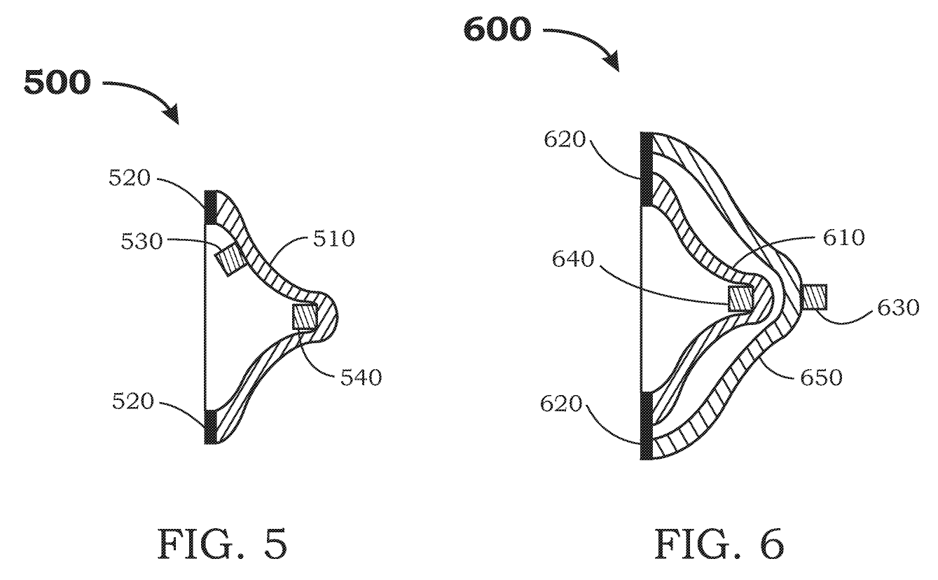

FIG. 5 is a side view illustrating one embodiment of a chest-piece 500 which combines an acoustic transducer 530 with an acoustic sensor 540 in a bell shaped housing 510, the chest-piece 500 useful with the auscultation device of the present invention. Such a devise may be utilized in an echo type method as illustrated in FIG. 1B. Acoustic transducer 530 and an acoustic sensor 540 may be piezos; however traditional microphone and speaker arrangements may also be utilized.

The acoustic sensor 540 may be sensitive to sound frequencies between 10 Hz to 500 Hz as well as frequencies generated by the acoustic transducer 530. Thus the acoustic sensor 540 may provide auscultation as well as attenuation measurement for calibration. Alternatively, in some embodiments, the acoustic transducer 530 generates sound waves in the MHz range, and it may be more desirable for the acoustic sensor 540 may be comprised of multiple sensors to cover the range of physiological and generated sound waves. Thus one benefit of a separate acoustic sensor 540 may be a more sensitive sensory capability across a greater frequency range.

An additional benefit of separate acoustic transducer 530 and acoustic sensor 540 is the elimination of the sensory blindness that occurs during generation when a single transducer is utilized. As such, a chest-piece as illustrated generally at 500 may provide continuous, as well as pulse acoustic attenuation.

ECG sensor 520 may include a sealing membrane to ensure both electrical conduction and mechanical air seal for superior acoustic transmission. Sealing may also be accomplished by an ECG gel in combination with or in place of a sealing membrane.

FIG. 6 is a side view of another exemplary chest-piece 600 which includes an acoustic transducer 630 located in an outer annulus 650 combined with an acoustic sensor 640 located on an inner sensing bell 610, the chest-piece 600 useful with the auscultation device of the present invention.

The chest piece depicted generally at 600 provides the same functionalities as the one shown at FIG. 5; however, by separating the acoustic transducer 630 from the acoustic sensor 640 within separate bells there may be a reduction in interference from the acoustic transducer 630 signal and the acoustics received by the acoustic sensor 640. Again the acoustic sensor 640 may be a sensory array, enabled to sense across a wide range of sound frequencies.

ECG sensor 620 may include a sealing membrane to ensure both electrical conduction and mechanical air seal for superior acoustic transmission. Sealing may also be accomplished by an ECG gel in combination with or in place of a sealing membrane.

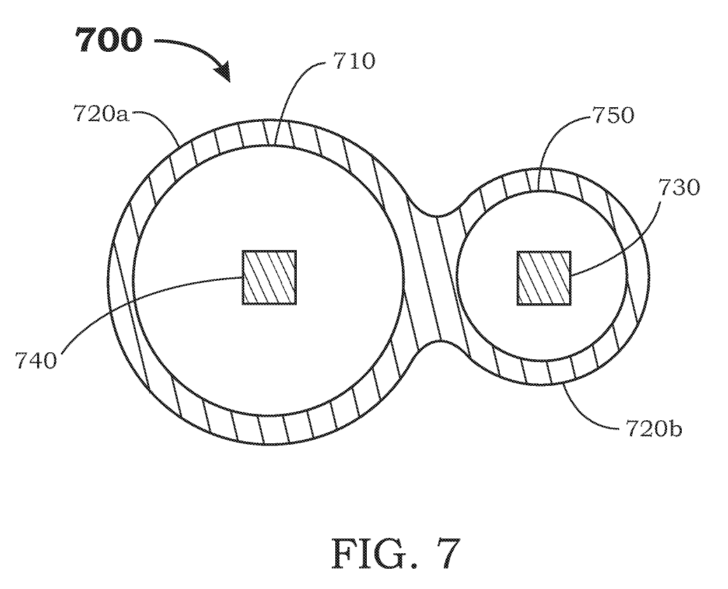

FIG. 7 is a bottom view illustrating yet another chest-piece 700 which includes an acoustic sensor 740 located in a sensing cavity 710 combined with an acoustic transducer 730 located in an attached auxiliary cavity 750. Cavities 710, 750 function as independent acoustic chambers to minimize cross-interference between transducer 730 and sensor 740. Optional sealing membrane 720a, 720b may be added to improve the acoustic properties of cavities 710, 750, respectively.

Although not illustrated, the Chest-Piece 700 may include an ECG sensor, which may include a sealing membrane to ensure both electrical conduction and mechanical air seal for superior acoustic transmission. Sealing may also be accomplished by an ECG gel in combination with or in place of a sealing membrane.

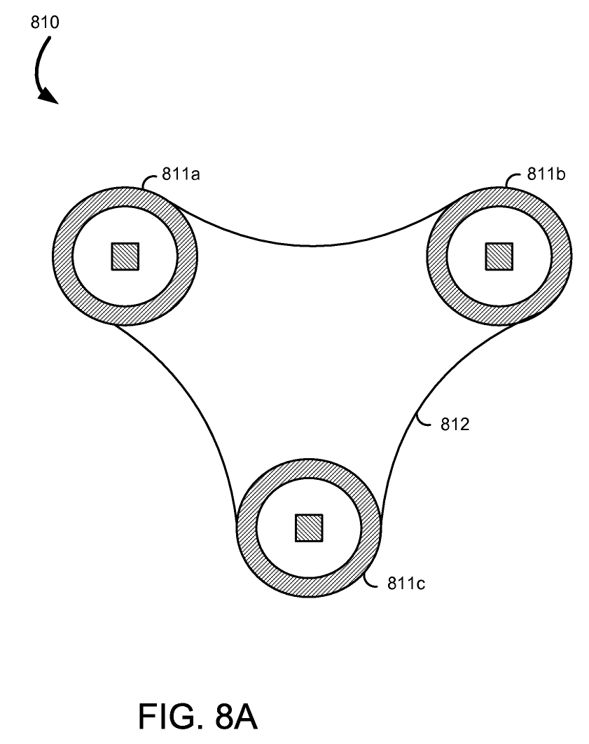

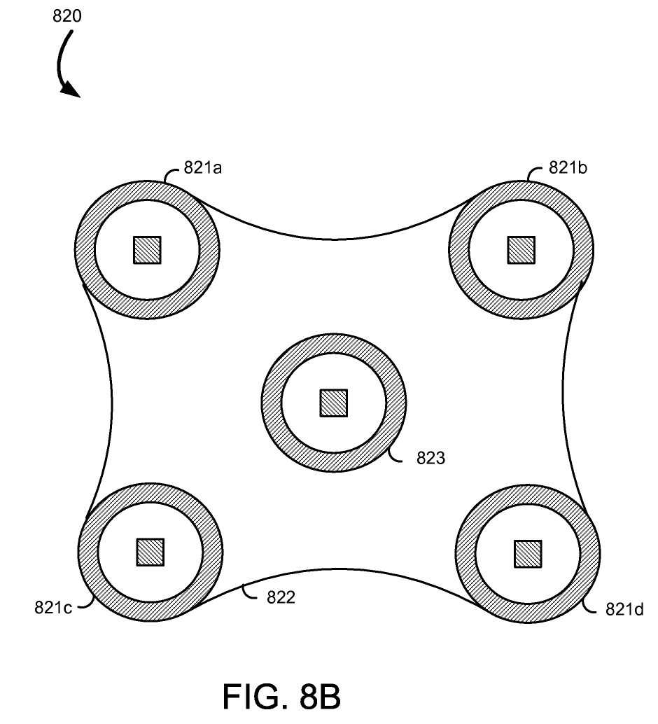

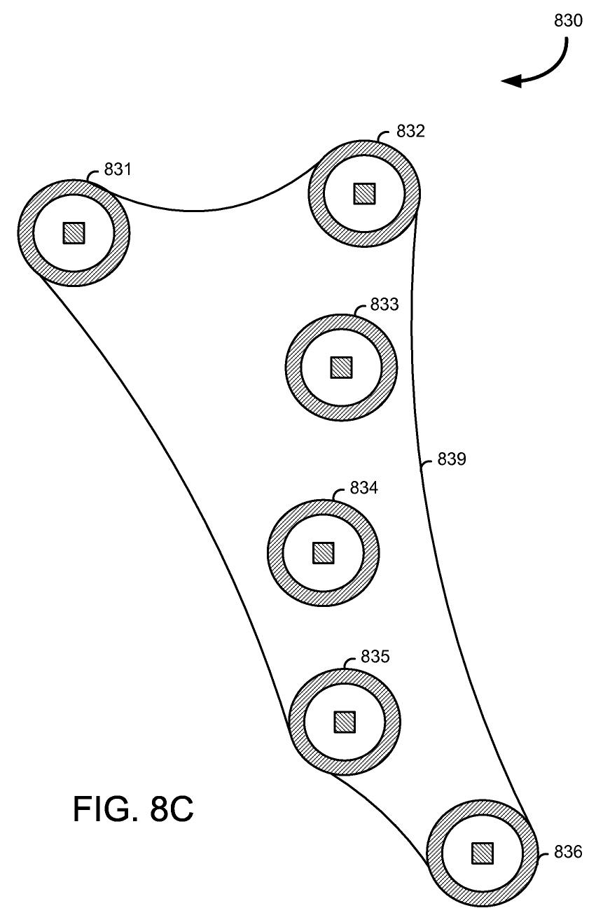

FIGS. 8A is a bottom view illustrating yet another chest-pad 810 which includes a triplet of Acoustic Sensors labeled 811a, 811b and 811c respectively. Acoustic Sensors 811a, 811b and 811c may be interconnected by a Webbing 812.

Webbing 812 may, in some embodiments, be a cloth mesh or plastic. Alternatively, Webbing 812 may be rigid in nature and include metal or plastics. In some embodiments, Webbing 812 may be connector rods of any suitable material. Webbing 812 functions to maintain the relative positions of the Acoustic Sensors 811a, 811b and 811c to one another.

Acoustic Sensors 811a, 811b and 811c may be arranged in an isosceles triangular fashion. Alternate orientations may additionally be utilized as is desired. In some embodiments, Acoustic Sensors 811a, 811b and 811c may be bell shaped sensor pads, with a microphone or piezo sensor in the vertex of the bell. Additionally, Acoustic Sensors 811a, 811b and 811c may include ECG functionality.

Acoustic Sensors 811a, 811b and 811c may, in some embodiments, additionally provide an active signal through a transducer. In other embodiments, a separate transducer may be utilized to generate the active acoustic signals.

Moreover, perceived signals by the Acoustic Sensors 811a, 811b and 811c may enable depth and location triangulation for internal structures when utilizing echo signals.

In some embodiments, the Chest Pad 810 may be designed in variant sizing for separate body sizes and types. In some embodiments, the Webbing 812 may be elastic as to increase wearer comfort and enable a singular device to be utilized by a wide gamut of individuals.

FIGS. 8B is a bottom view illustrating yet another chest-pad 820 which includes a quintuplet of Acoustic Sensors labeled 821a, 821b, 821c, 821d and 823 respectively. Acoustic Sensors 821a, 821b, 821c, 821d and 823 may be interconnected by a Webbing 822. In the present design Acoustic Sensors 821a, 821b, 821c and 821d are oriented in a square geometry around a central Acoustic Sensor 823. Alternate orientations may additionally be utilized as is desired. The central Acoustic Sensor 823 may, in some embodiments, provide a transducer. Additional Acoustic Sensors 821a, 821b, 821c, 821d and 823 may, in some embodiments, additionally provide an active signal through a transducer. In other embodiments, a separate transducer may be utilized to generate the active acoustic signals.

As previously discussed, Webbing 822 may, in some embodiments, be a cloth mesh or plastic. Alternatively, Webbing 822 may be rigid in nature and include metal or plastics. In some embodiments, Webbing 822 may be connector rods of any suitable material. Webbing 822 functions to maintain the relative positions of the Acoustic Sensors 821a, 821b, 821c, 821d and 823 to one another.

In some embodiments, Acoustic Sensors 821a, 821b, 821c, 821d and 823 may be bell shaped sensor pads, with a microphone or piezo sensor in the vertex of the bell. Additionally, Acoustic Sensors 821a, 821b, 821c, 821d and 823 may include ECG functionality.

Moreover, perceived signals by the Acoustic Sensors 821a, 821b, 821c, 821d and 823 may enable depth and location triangulation for internal structures when utilizing echo signals.

As previously discussed, in some embodiments, the Chest Pad 820 may be designed in variant sizing for separate body sizes and types. In some embodiments, the Webbing 822 may be elastic as to increase wearer comfort and enable a singular device to be utilized by a wide gamut of individuals.

FIGS. 8C is a bottom view illustrating yet another chest-pad 830 which includes a sextet of Acoustic Sensors labeled 831, 832, 833, 834, 835 and 836 respectively. Acoustic Sensors 831, 832, 833, 834, 835 and 836 may be interconnected by a Webbing 839. In the present design Acoustic Sensors 831, 832, 833, 834, 835 and 836 are oriented at the anterior thoracic ECG positions V1, V2, V3, V4, V5 and V6 respectively, as shown in FIG. 2. The Webbing 839 ensures proper placement of the Acoustic Sensors 831, 832, 833, 834, 835 and 836 across the patients torso, and enables the application of a single pad for multiple readouts.

As previously discussed, Webbing 839 may, in some embodiments, be a cloth mesh or plastic. Alternatively, Webbing 839 may be rigid in nature and include metal or plastics. In some embodiments, Webbing 839 may be connector rods of any suitable material. Webbing 839 functions to maintain the relative positions of the Acoustic Sensors 831, 832, 833, 834, 835 and 836 to one another.

In some embodiments, Acoustic Sensors 831, 832, 833, 834, 835 and 836 may be bell shaped sensor pads, with a microphone or piezo sensor in the vertex of the bell. Additionally, Acoustic Sensors 831, 832, 833, 834, 835 and 836 may include ECG functionality.

Moreover, Acoustic Sensors 831, 832, 833, 834, 835 and 836 may, in some embodiments, additionally provide an active signal through a transducer. In other embodiments, a separate transducer may be utilized to generate the active acoustic signals.

Moreover, perceived signals by the Acoustic Sensors 831, 832, 833, 834, 835 and 836 may enable depth and location triangulation for internal structures when utilizing echo signals.

As previously discussed, in some embodiments, the Chest Pad 830 may be designed in variant sizing for separate body sizes and types. In some embodiments, the Webbing 839 may be elastic as to increase wearer comfort and enable a singular device to be utilized by a wide gamut of individuals.

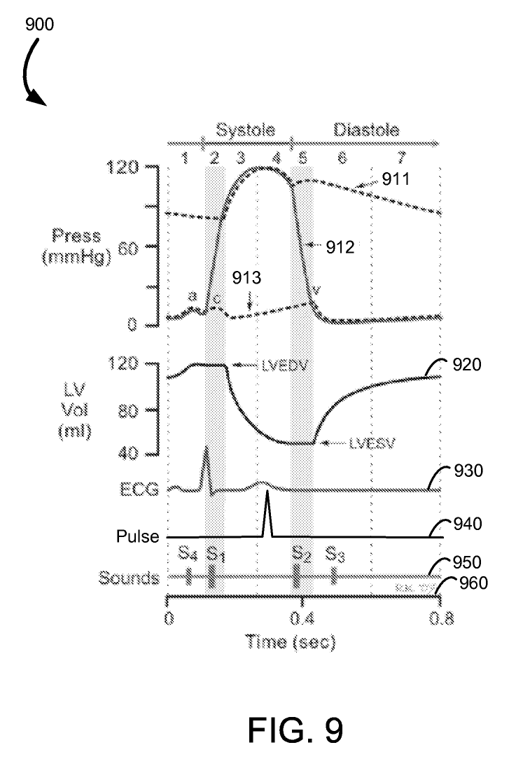

FIG. 9 shows an exemplary diagram of pressure, timing, blood volume and signals associated in a typical cardiac cycle, shown generally at 900.

The cardiac cycle diagram shown depicts changes in aortic pressure (AP) 911, left ventricular pressure (LVP) 912, left atrial pressure (LAP) 913, left ventricular volume (LV Vol) 920, acoustic echo Pulse 940 and heart sounds 950 during a single cycle of cardiac contraction and relaxation. These changes are related in time to the electrocardiogram.

Typically aortic pressure is measured by inserting a pressure catheter into the aorta from a peripheral artery, and the left ventricular pressure is obtained by placing a pressure catheter inside the left ventricle and measuring changes in intraventricular pressure as the heart beats. Left arterial pressure is not usually measured directly, except in investigational procedures. Ventricular volume changes can be assessed in real time using echocardiography or radionuclide imaging, or by using a special volume conductance catheter placed within the ventricle.

A single cycle of cardiac activity can be divided into two basic stages. The first stage is diastole, which represents ventricular filling and a brief period just prior to filling at which time the ventricles are relaxing. The second stage is systole, which represents the time of contraction and ejection of blood from the ventricles.

The Pulse 940 shown is intended to be exemplary in nature. The Pulse 940 may be 10 to 100 microseconds in length. In some embodiments, longer pulses may be utilized. The diagram illustrates a longer Pulse 940 for viewing ease. In yet other embodiments, continuous acoustic signals may be supplied by the acoustic transducer. Additionally, the Pulse 940 may be varied in time across the cardiac cycle as to interleave the Pulse 940 and heart sounds.

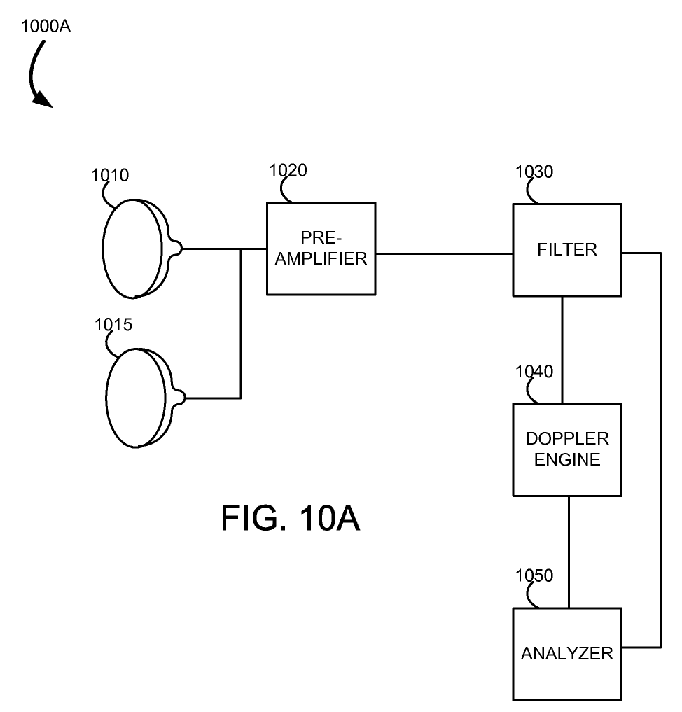

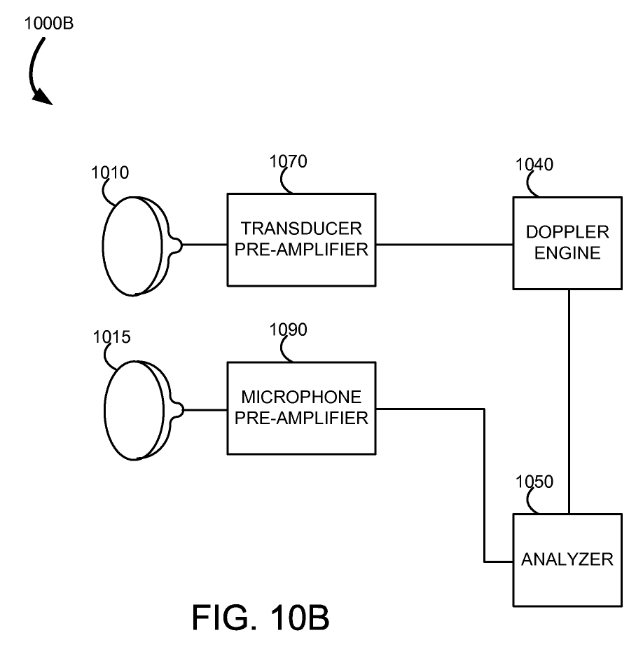

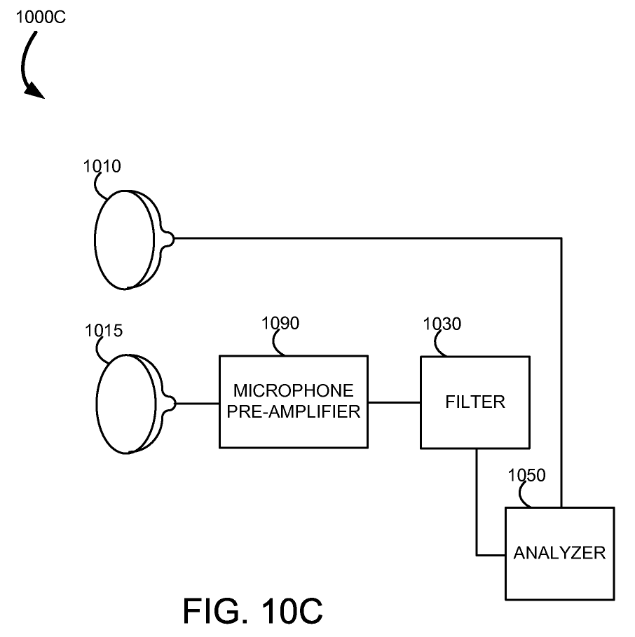

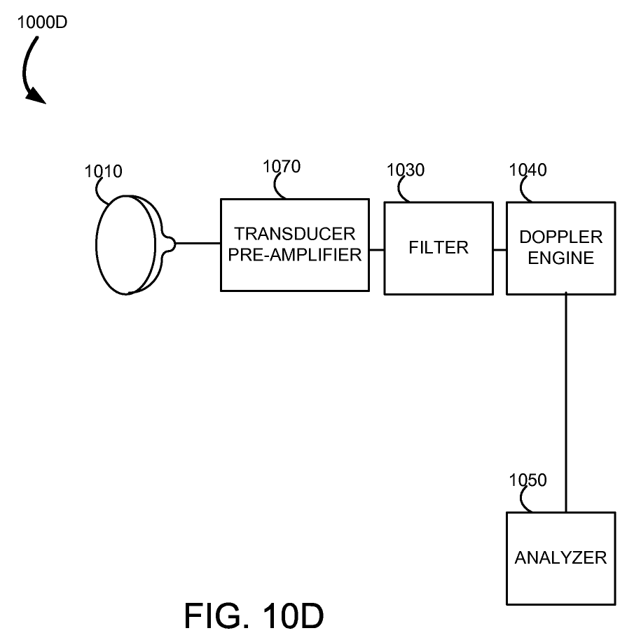

FIGS. 10A through 10D provide exemplary functional diagrams of the auscultatory device. Additional embodiments are possible, and it is intended that the spirit of these additional embodiments is included in the exemplary embodiments.

FIG. 10A shows a functional block diagram of one embodiment of the auscultatory device shown generally at 1000A. The Acoustic Transducer 1010 may be any of the sensory devices illustrated in FIGS. 3A to 7, as well as any sensory design as is known by those skilled in the art. The Acoustic Transducer 1010 may couple to a Pre-amplifier 1020. An Acoustic Sensor 1015 may be any acoustic sensor designed to be responsive to heart sounds, such as a Stethoscope 120. The Acoustic Sensor 1015 may likewise couple to the Pre-amplifier 1020. In some embodiments, the Acoustic Sensor 1015 and Acoustic Transducer 1010 may be housed within the same unit. Additionally, in some embodiments, a single sensor may incorporate both the Acoustic Sensor 1015 and Acoustic Transducer 1010.

The Pre-amplifier 1020 may amplify the source signal to line signal levels. Additional equalizing and tone control may be performed by the Pre-amplifier 1020 as well. In some embodiments, where the echo signal received from the Acoustic Transducer 1010 far outweighs the heart signals from the Acoustic Sensor 1015, additional protective circuitry may be utilized in order to preserve the heart sound signals.

The Pre-amplifier 1020 couples to a Filter 1030, which is enabled to separate the signals relating to heart sounds from those received from the echo of the generated acoustic signals. As previously discussed, Heart sounds are typically low in frequency, e.g. typically 10 to 500 Hz the generated acoustic signals may be in the MHz range. As such high pass and low pass filters may easily distinguish between sounds originating from the heart, and those echoing from the generated signals from the acoustic transducer.

The Filter 1030 may be coupled to a Doppler Engine 1040 and an Analyzer 1050. The Doppler Engine 1040 may, in some embodiments, receive the echo signals separated by the Filter 1030, while the heart sounds are sent directly to the Analyzer 1050. The Doppler Engine 1040 may be enabled to deduce the speed at which the valve leaflet is moving with respect to the sound wave by detecting Doppler shifting. Alternatively, another way to deduce the speed is to measure the distance traversed by the moving leaflet, and knowing the time interval between the two measurements and computing leaflet speed. In such embodiments the Doppler Engine 1040 may be unnecessary. The former involves more sophisticated electronics; the latter is simpler in implementation but may be less precise. Additional methods of determining valve leaflet speed may be utilized as is known by those skilled in the art.

The Doppler Engine 1040 also allows for blood flow to be detected and further helps to characterize any heart sound component caused by regurgitant jet. Additionally, Doppler processing increases the accuracy and robustness of determining the spatial (which valve) and temporal (systole or diastole) origin of a murmur.

The Analyzer 1050 may provide display and analysis of the received signals. Such analysis includes, but is not limited to S1/S2 sound ratios and heart sound calibration utilizing the ratio of S1 and the attenuated sound (Sc), the intensity ratio (S1/Sc).

FIG. 10B shows a functional block diagram of another embodiment of the auscultatory device shown generally at 1000B. The Acoustic Transducer 1010 may be any of the sensory devices illustrated in FIGS. 3A to 7, as well as any sensory design as is known by those skilled in the art. The Acoustic Transducer 1010 may couple to a Transducer Pre-amplifier 1070. An Acoustic Sensor 1015 may be any acoustic sensor designed to be responsive to heart sounds, such as a Stethoscope 120. The Acoustic Sensor 1015 may be couple to the Microphone Pre-amplifier 1090. In some embodiments, the Acoustic Sensor 1015 and Acoustic Transducer 1010 may be housed within the same unit.

The Transducer Pre-amplifier 1070 may amplify the perceived pulse echo signal to a line signal levels. Additional equalizing and tone control may be performed by the Transducer Pre-amplifier 1070 as well. Likewise, the Microphone Pre-amplifier 1090 may amplify the perceived heart sound signal to a line signal levels. Additional equalizing and tone control may be performed by the Microphone Pre-amplifier 1090 as well. The utilization of two channels dedicated to heart sounds and pulse echo signals separately eliminates the requirement for filters.

The Transducer Pre-amplifier 1070 may be coupled to a Doppler Engine 1040. As previously stated, the Doppler Engine 1040 may be enabled to deduce the speed at which the valve leaflet is moving with respect to the sound wave by detecting Doppler shifting. Alternatively, as previously discussed, alternative methods for determining valve leaflet speed may be utilized.

The Doppler Engine 1040 also allows for blood flow to be detected and further helps to characterize any heart sound component caused by regurgitant jet. Additionally, Doppler processing increases the accuracy and robustness of determining the spatial (which valve) and temporal (systole or diastole) origin of a murmur.

The Microphone Pre-amplifier 1090 and the Doppler Engine 1040 couple to the Analyzer 1050 which may provide display and analysis of the received signals. Such analysis includes, but is not limited to S1/S2 sound ratios and heart sound calibration utilizing the ratio of SI and the attenuated sound (Sc), the intensity ration (S1/Sc).

FIG. 10C shows a functional block diagram of yet another embodiment of the auscultatory device shown generally at 1000C. The Acoustic Transducer 1010 may be any acoustic generation device, such as speaker or piezo, as is known by those skilled in the art. An Acoustic Sensor 1015 may be any acoustic sensor designed to be responsive to heart sounds and the acoustic signal generated by the Transducer 1010, such as a Stethoscope 120. The Acoustic Sensor 1015 may be couple to the Microphone Pre-amplifier 1090. In some embodiments, the Acoustic Sensor 1015 and Acoustic Transducer 1010 may be housed within the same unit.

The Microphone Pre-amplifier 1090 may amplify the perceived heart sound signal and tansduced signal to a line signal levels. Additional equalizing and tone control may be performed by the Microphone Pre-amplifier 1090 as well. A Filter 1030 may separate the perceived heart sound signal from the transduced signal. Alternatively, in some embodiments, time interleaving may be utilized in order to temporally separate heart signals from transduced signals.

The Transducer 1010 and the Filter 1030 couples to the Analyzer 1050 which may provide display and analysis of the received signals. Such analysis includes, but is not limited to S1/S2 sound ratios and heart sound calibration utilizing the ratio of S1 and the attenuated sound (Sc), the intensity ratio (S1/Sc).

FIG. 10D shows a functional block diagram of yet another embodiment of the auscultatory device shown generally at 1000D. The Acoustic Transducer 1010 may be any of the sensory devices illustrated in FIGS. 3A to 7, as well as any sensory design as is known by those skilled in the art. In this and similar embodiments the Transducer 1010 may both generate a pulse echo as well as provides sensory ability. The Acoustic Transducer 1010 may couple to a Transducer Pre-amplifier 1070. Transducer 1010 may be designed to be responsive to heart sounds as well as the generated pulse echo.

The Transducer Pre-amplifier 1070 may amplify the perceived pulse echo signal and heart sound signal to a line signal levels. Additional equalizing and tone control may be performed by the Transducer Pre-amplifier 1070 as well. A Filter 1030 may separate the perceived heart sound signal from the tansduced signal. Alternatively, in some embodiments, time interleaving may be utilized in order to temporally separate heart signals from transduced signals.

The Filter 1030 may be coupled to a Doppler Engine 1040. As previously stated, the Doppler Engine 1040 may be enabled to deduce the speed at which the valve leaflet is moving with respect to the sound wave by detecting Doppler shifting. Alternatively, as previously discussed, alternative methods for determining valve leaflet speed may be utilized.

The Doppler Engine 1040 also allows for blood flow to be detected and further helps to characterize any heart sound component caused by regurgitant jet. Additionally, Doppler processing increases the accuracy and robustness of determining the spatial (which valve) and temporal (systole or diastole) origin of a murmur.

The Doppler Engine 1040 couples to the Analyzer 1050 which may provide display and analysis of the received signals. Such analysis includes, but is not limited to S1/S2 sound ratios and heart sound calibration utilizing the ratio of S1 and the attenuated sound (Sc), the intensity ratio (S1/Sc), speed and motion analysis and localization of sound sources.

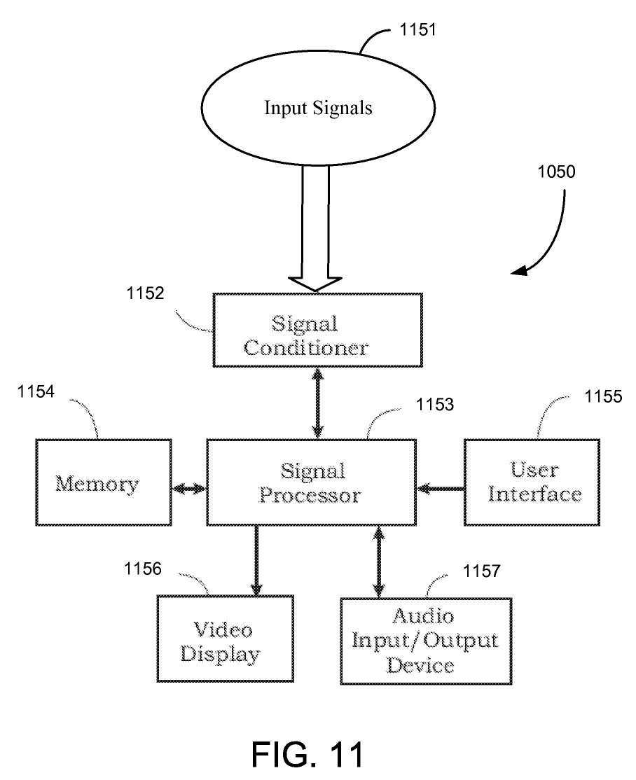

FIG. 11 shows an illustration of a functional block diagram for the Analyzer 1050 in accordance with an embodiment of the present invention. Analyzer 1050 includes a Signal Conditioner 1152, Signal Processor 1153, Memory 1154, User Interface 1155, Video Display 1156 and Acoustic Input/Output Device 1157.

Input Signals 1151 are received from the Doppler Engine 1040, Filter 1030 and Microphone Pre-Amplifier 1090. Such raw Input Signals 1151 are processed through a Signal Conditioner 1152. Conditioned signals may then be analyzed by the Signal Processor 1153. Signal Processor 1153 may couple to Memory 1154, User Interface 1155, Video Display 1156 and Acoustic Input/Output Device 1157.

Memory 1154 can be fixed or removal memory, and combinations thereof Examples of suitable technologies for memory 1154 include solid-state memory such as flash memory, or a hard disk drive.

User interface 1155 can be a keypad, a keyboard, a thumbwheel, a joystick, and combinations thereof. Video display 1156 can be an LCD screen, or can be an LED display or a miniature plasma screen. It is also possible to combine video display 1156 with user interface 1155 by use of technologies such as a touch screen. Contrast and brightness control capability can also be added to display 1156.

Acoustic input/output (I/O) device 1157 includes a microphone, and speakers, earphones or headphones, any of which can be internal or external with respect to Analyzer 1050. It is also possible to use wireless acoustic I/O devices such as a Bluetooth-based headset. Volume control may also be provided.

Logical couplings of these components may be otherwise organized as is advantageous. Additionally, alternate or additional components may be included in the Analyzer 1050.

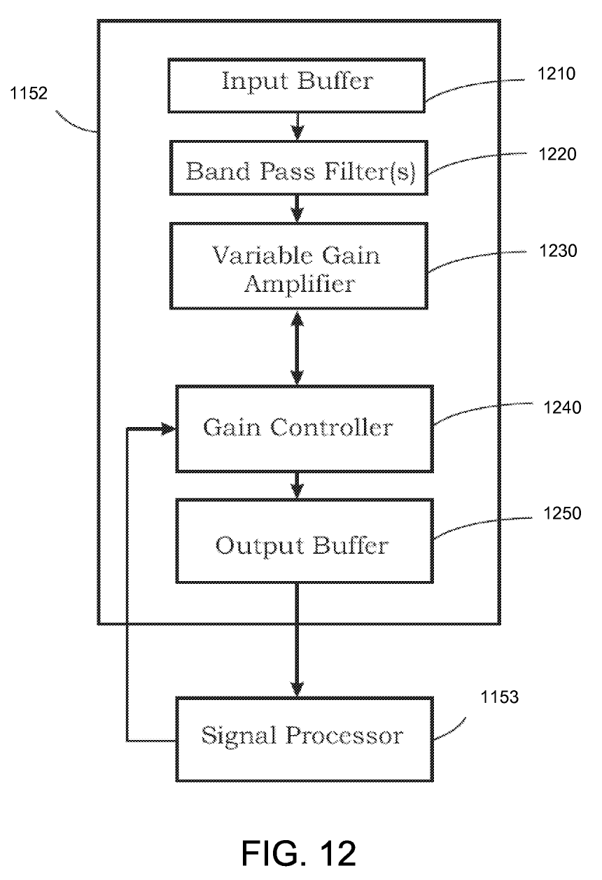

FIG. 12 provides a detailed block diagram illustrating heart sound Signal Conditioner 1152 which includes an Input Buffer 1210, one or more Band Pass Filter(s) 1220, a Variable Gain Amplifier 1230, a Gain Controller 1240 and an Output Buffer 1250. Output buffer 1250 is coupled to Signal Processor 1153 which in turn is coupled to Gain Controller 1240.

In some embodiments, Filter 1220 is a 4th order Butterworth pass band of 5 Hz to 2 kHz which limits the analysis of the heart sound signal to frequencies less than 2 kHz, thereby ensuring that all frequencies of the heart sounds are faithfully captured and at the same time eliminating noise sources that typically exist beyond the pass band of Filter 1220. Of course additional Filters 1220 may be utilized as is desired.

Variable Gain Amplifier 1230 of Signal Conditioner 1152 serves to vary the signal gain based on a user-selectable input parameter, and also serves to ensure enhanced signal quality and improved signal to noise ratio. The conditioned heart sound signal after filtering and amplification is then provided to Signal Processor 1153 via Output Buffer 1250.

Additional signal conditioning components may be incorporated into the Signal Conditioner 1152 as is desired. For example, in some embodiments, a component for eliminating low amplitude noise signals may be utilized.

### Self Calibration

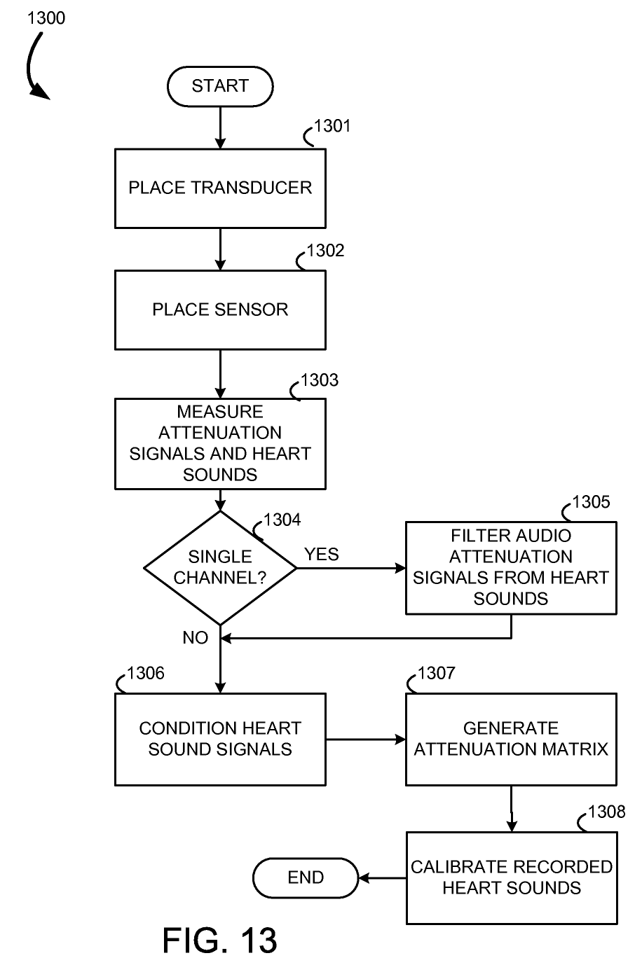

FIGS. 13 to 15 provide methods and processes for the calibration of heart sound measurements by use of an active transduction signal. Such a signal may be measured to produce attenuation values and subsequent heart sound calibrations. Heart sound calibration has diagnostic use, and provides an ability to perform cross-patient heart sound analyses.

FIG. 13 shows an exemplary process for self calibration of heart signals utilizing an embodiment of the auscultatory device, shown generally at 1300. Such a process may be performed automatically by the auscultatory device, without need of user input. Such a process may equalize heart sounds from a range of patients. Additionally, calibrated heart signals may be utilized in a range of subsequent diagnostic processes, such as Ejection Fraction determination.

In some embodiments, there are two ways to calibrate S1, each with its own advantages and disadvantages. The first includes calibrating S1 with S2. The advantage of this method is that each patient will calibrate him/herself, since the body equally attenuates both sounds and there is no additional need to work out different attenuations for different people. A simple comparison of a patient's S1 intensity to their S2 intensity may be utilized to produce meaningful diagnostic ratios. The disadvantage of this method is that S2 itself may be affected by a heart condition and may be unsuitable.

Secondly, calibration of the S1 may be performed by utilizing the attenuation values recorded. In some embodiments, multiple tones may be utilized, at various frequencies in the first heart sound spectrum. The advantage of this method is that the attenuation of the tones should be representative on each subject of sound attenuation in their body. There is no bias regarding their cardiac health, as is the case with calibration by S2. In some embodiments, the transmission tones are just simple tones; however more complex attenuation signals may be utilized.

The process begins at step 1301 where the transducer is placed upon the patient at the Transducing Location 141. Any transducer disclosed in FIGS. 3 to 8c may be utilized. In some embodiments, such transducers produce a constant active signal during calibration. A sensor may be placed at the Sensing Location 131 at step 1302. The sensor may receive signals that pass through the patient's body. These received signals are measured at step 1303. As addressed earlier, the transduced signals may be within physiological frequency ranges. Additional frequencies, steeped frequencies and variable frequencies may also be utilized. A single sensor may be utilized to measure both generated attenuation signal as well as patient heart sounds. Alternatively, additional sensors may be utilized to measure heart sounds and attenuation signals. Sensor(s) responsive range is calibrated to be sensitive to attenuation signal range and physiological sound ranges.

At step 1304, a determination is made as to whether heart sounds and attenuation signals are on the same channel. Such is the case when attenuation signal and heart sounds are perceived by a common sensor. If these signals share a single channel, the signals may be filtered at step 1305. Filtering may be performed by band pass filtering, in the instances where attenuation signal is of a separate frequency range than heart sounds. Alternatively, filtering may include a very narrow band pass filter for the attenuation frequency when the attenuation signal is within a physiological range. The signal is then conditioned at step 1306.

If, at step 1304, the attenuation signal and the heart sounds are on separate channels, then the signal is conditioned at step 1306. Separate channels for the heart signals and attenuation signals is achieved when separate frequency ranges are utilized for the attenuation signal as compared to the heart sound frequency, and separate sensors are utilized for the measuring of the respective signals. The sensors may, in some embodiments, be responsive to the particular frequency range they are measuring thereby providing an intrinsic filtering.

After signal conditioning, the process proceeds to step 1307, where an attenuation matrix is generated. To generate the matrix, the signal amplitude for each transducer/sensor location is compiled.

Then at step 1308, the measured heart sounds may be calibrated by using the attenuation matrix. The S1 may be calibrated by the use of any combination of the values in the attenuation matrix.

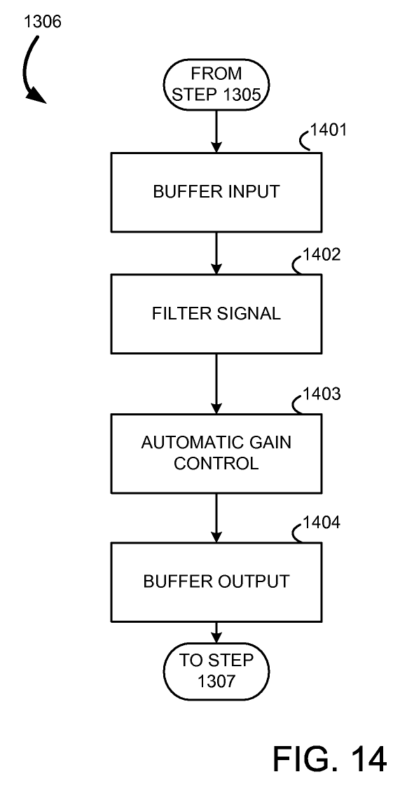

FIG. 14 shows an exemplary process for signal conditioning of heart signals utilizing an embodiment of the auscultatory device, shown generally at 1306. Signal conditioning may occur at the Signal Conditioner 1152.

The process begins from step 1305 from FIG. 13. The process then proceeds to step 1401 where the input signal is buffered. Buffering occurs at the Input Buffer 1201. Then, at step 1402, the signal may undergo additional filtering. The filtering operations may involve simple filters, for example a straightforward analog Butterworth nth order bandpass/lowpass/highpass filter. It is conceivable that wavelet operations, which by their nature divide up the signal into various frequency bands, can also be used to carry out measurements on the heart sound signal. Additional filtering techniques may be employed as is known by those skilled in the art. Filtering may occur at the Band Pass Filter(s) 1202.

The process then proceeds to step 1403 where gain may be automatically controlled. A Variable Gain Amplifier 1203 in conjunction with the Gain Controller 1204 may effectuate automatic gain control.

The process then proceeds to step 1404 where the output is buffered. The Output Buffer 1205 may perform this operation. Additional signal conditioning steps may be performed as is known by those skilled in the art. The process then ends by proceeding to step 1307.

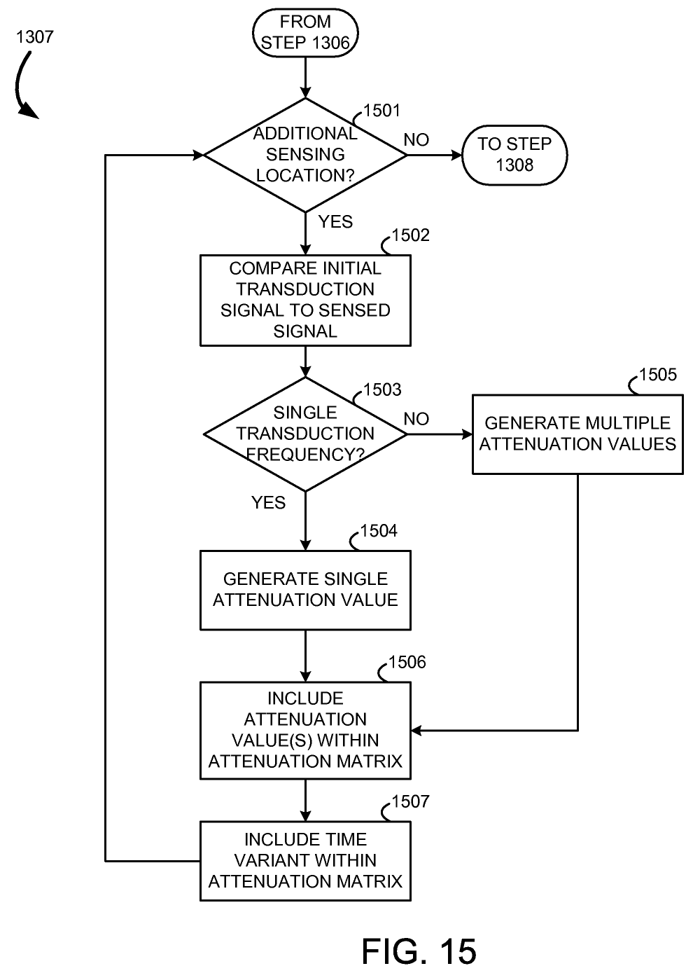

FIG. 15 shows an exemplary process for generating the attenuation matrix utilizing an embodiment of the auscultatory device, shown generally at 1307. The use of an attenuation matrix is but one suitable method of representing attenuation signal data for use with calibration. As such, the present method is intended to be exemplary in nature. No limitations upon the present invention are suggested by the disclosure of attenuation matrix generation. Moreover, additional representations, such as a single attenuation value, an attenuation value list or three dimensional attenuation value matrices may be utilized.

The process begins from step 1306. Then at step 1501 an inquiry is made whether an additional sensing location is desired. If at step 1306 an additional sensing location is desired, then the process proceeds to step 1502, where the known initial transduction signal is compared to the perceived attenuation signal. The initial transduction signal may, in some embodiments, include a constant sinusoidal sound signal. Alternative sound waveforms, frequencies and durations may be utilized as is desired. The difference between the known initial transduction signal and the perceived attenuation signal provides information about internal structures along the sound wave path.

Then at step 1503, an inquiry is made as to whether the transduction signal was a single frequency signal. If so, then at step 1504 a single attenuation value may be generated. The single attenuation value may then be added to an attenuation matrix in step 1506.

Else, if at step 1503, the initial transduction signal was not of a single frequency, then the process proceeds to step 1505 where multiple attenuation values are generated. The multiple attenuation values may then be added to an attenuation matrix in step 1506.

Then in step 1507, a time variant value may be added to the matrix. The time variant value is the time differential between signal transduction and perceived attenuation signal measurement.

The process then proceeds back to step 1501, where an inquiry is made whether an additional sensing location is desired. In this way the process will be repeated for each sensing location desired. Attenuation values for each sensing location may be compiled into the attenuation matrix. Once all sensing locations have been measured the process ends.

In this way heart sounds may be calibrated for by utilizing an active transduction signal that passes through the patient's chest cavity. Additional methods for heart sound calibration may additionally be utilized, including both invasive and non-invasive procedures.

### Pulsed Echo

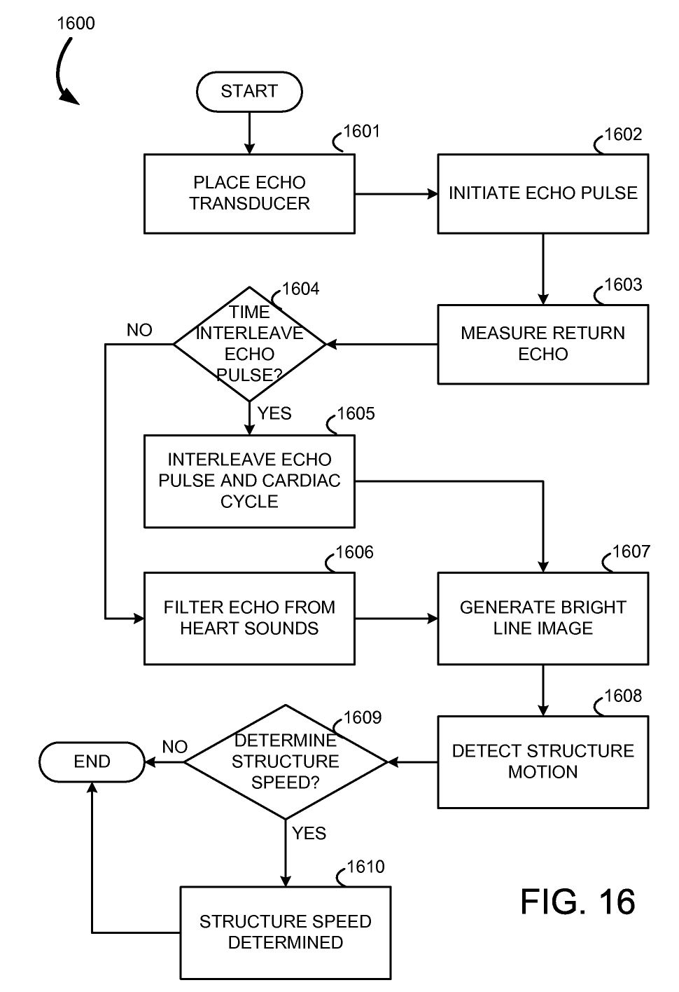

FIGS. 16 to 18 further illustrate methods for pulsed echo cartographic analysis. Pulsed echo refers to the usage of pulsed acoustics to provide a reflective “image” of internal structures. In some embodiments, the echo pulse may be of higher frequencies as to provide adequate resolutions. The ability to sense structure motion, location and speed of motion makes the pulsed echo of particular use in identifying pathologies such as a faulty valve.

FIG. 16 shows an exemplary process for pulsed echo utilizing an embodiment of the auscultatory device, shown generally at 1600. The process begins at step 1601 where the pulsed echo transducer is placed in the transducer position on the patient's torso. Then, at step 1602, an echo pulse is induced. The echo pulse, in some embodiments, may be a few microseconds up to few tens of microseconds in duration. Operating in MHz range provides adequate resolution. Echo pulses may be repeated as necessary.

At step 1603 the return echo is measured. Then, at step 1604, an inquiry is made whether to utilize time interleaving. If time interleaving is desired then the process proceeds to step 1605 where echo pulses and cardiac signals are interleaved as to minimize the potential loss of signal data. Time interleaving separates heart signals from echo pulse temporally, thereby removing the need for additional filtering. Time interleaving may additionally be useful when the echo pulse saturates the received signals. Then at step 1607 a bright line image is generated. The bright line image is a representation of the structures encountered by the pulse echo.

Else if at step 1604 time interleaving is not desired the process then proceeds to step 1606, where the heart signals are filtered from the echo signals. Since, in some embodiments, the echo pulse is of much higher frequency than heart sounds, a simple high pass filtering may be utilized to separate heart signals from the echo pulse. Then at step 1607 a bright line image is generated. The bright line image is a representation of the structures encountered by the pulse echo.

Then at step 1608 structure motion is identified. An inquiry is made if moving structure speed is to be determined at step 1609. In some embodiments, speed of moving structures may be automatically generated. In other embodiments, speed determination may be performed on a case by case basis. In such embodiments the user physician may select a mode for speed capture on the auscultatory device. If speed of the moving structure is desired the process proceeds to step 1610 where the structure speed is identified. Typical structures which speed may be measured includes heart valve leaflet closure rates, blood flow, heart wall constriction or any additional moving structure. After structure speed is determined the process ends. Else, if at step 1609 structure speed is not a required measurement, the process ends.

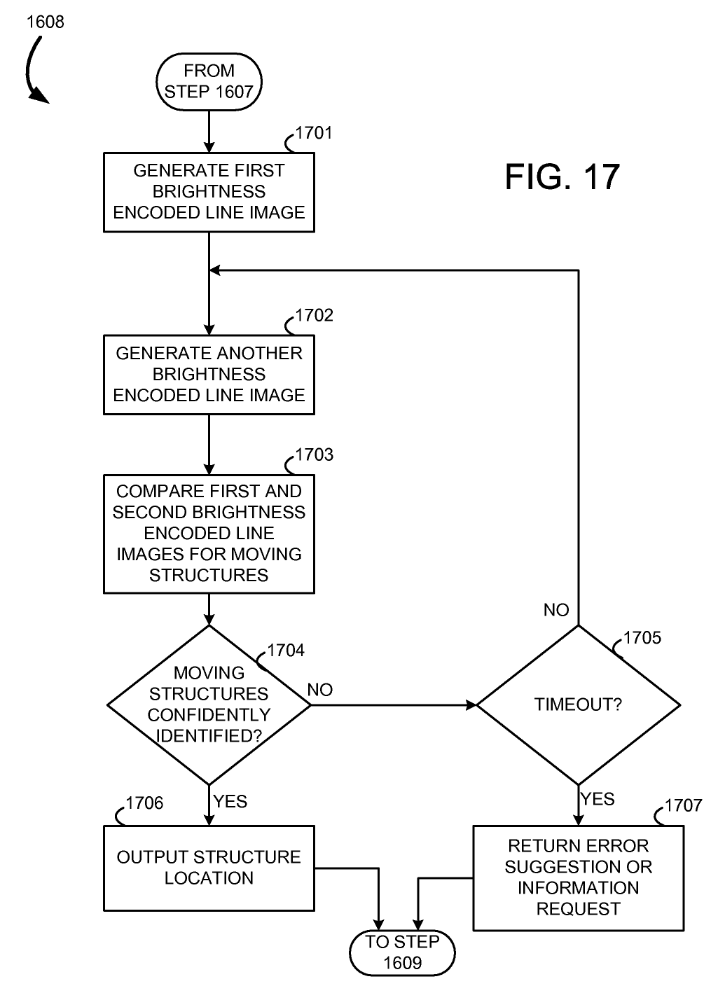

FIG. 17 shows an exemplary process for motion detection in pulsed echo utilizing an embodiment of the auscultatory device, shown generally at 1608. A brightness line image generated from the received acoustic signals provides a representation for the structures along the acoustic path. By maintaining a fixed acoustic path, and repeatedly sensing the structures, motion may be identified and tracked. A heart valve is in motion with respect to the patient's chest wall, thus the distance of the valve to the chest wall may be deduced. Such a deduction may accurately be used to enable the calibration of the heart sound of that particular patient to his chest size or attenuation characteristics (the amount of subcutaneous fat, for example).

Motion analysis helps to orient the heart sound to the particular valve as indicated by the motion trace and can achieve better isolation of particular disease signature of the heart sound associated with that particular valve.

The process begins from step 1607. At step 1701 a first brightness encoded image is generated. This first image is generated with the sensor fixed to the patient's chest. Thus the image provided is stationary in relation to patient's chest wall. Then at step 1702 another brightness encoded image is generated. Likewise, this additional image is generated with the sensor fixed to the patient's chest. Thus the image provided is stationary in relation to patient's chest wall. The two images are compared for moving structures at step 1703. Since both images “look” at the same space related to the patient's chest wall, discrepancies between the two brightness encoded images is a result of movement of the a structure. Additionally, pulse echo timing and orientation may additionally provide structure location information. Thus, the moving structures location may be likewise identified.

At step 1704 an inquiry is made whether the moving structure is adequately identified. A statistical analysis of confidence levels, as measured by a threshold, may be utilized to determine this. For example, if the auscultatory device is calibrated such that a greater than 75% identification of moving structures is required, and the brightness encoded images identify a moving structure 50% of the time the auscultatory device may determine that the structure is not adequately identified. In such a circumstance, the process then proceeds to step 1705 where an inquiry is made whether moving structure identification has timed out. If the process has not timed out, then the process may return to step 1702 where an additional brightness encoded image is generated in an attempt to clarify the identification. The process then continues the cycle of comparison, confidence inquiry, etc.

Else, if at step 1705 the process for determining the moving structure has timed out then the process proceeds to step 1707, where an error message is generated. Such an error message may provide either an information request or suggestion. For example, if the sensor is not pointing in a stable fashion due to hand motion etc, it may indicate repositioning or provide feedback to the user and likewise indicate when the sensor is pointing accurately at the moving structure. The process then ends by proceeding to step 1609.

Otherwise, if at step 1704 the moving structure is adequately identified then the process may output the moving structure's location at step 1706. The process then ends by proceeding to step 1609.

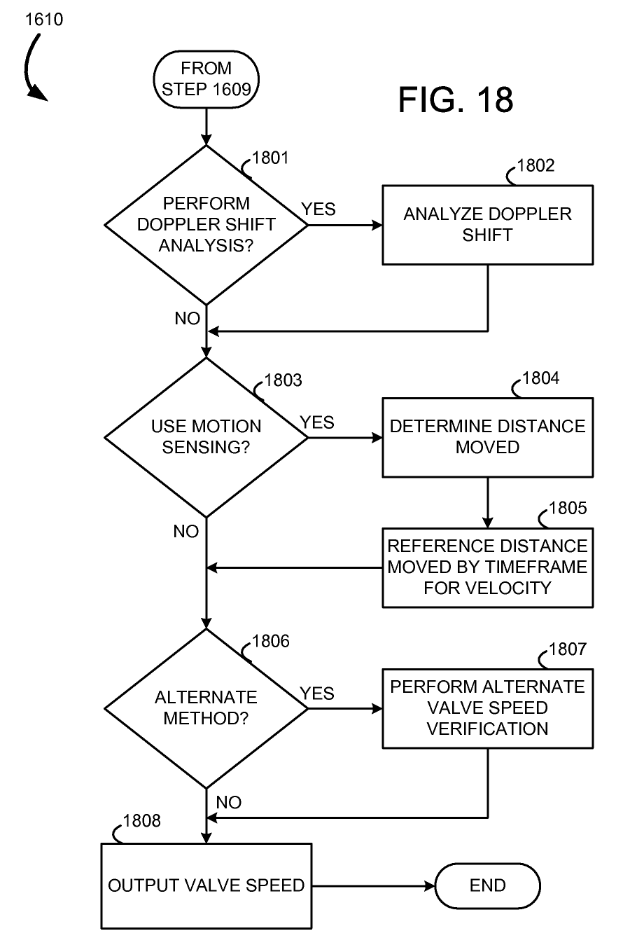

FIG. 18 shows an exemplary process for structure speed detection in pulsed echo utilizing an embodiment of the auscultatory device, shown generally at 1610. The illustrated method includes utilizing a motion trace, Doppler shift detection and alternate methods. In some embodiments, there may be limitations on hardware available, such as Doppler processors. In these embodiments the available hardware may dictate speed determination decisions.

The process begins from step 1609. Then at 1801 an inquiry is made whether to perform a Doppler shift analysis. If a Doppler shift analysis is desired then the process proceeds to step 1802 where the shift analysis is performed. As the pulse reflects from a moving structure the return echo will have shifted frequency as related to the speed of the moving structure. A Doppler Engine 1040 may measure the amount of frequency shift in order to determine structure speed. The process then progresses to step 1803 where an inquiry is made whether to determine structure speed by motion tracking.

Else, if at step 1801 a Doppler shift analysis is not performed, then the process progresses to step 1803 where an inquiry is made whether to determine structure speed by motion tracking. Motion tracking for speed determination is simpler than Doppler analysis and requires less hardware, however it tends to be less precise. In some embodiments, motion tracking may be performed in conjunction with Doppler analysis for speed confirmation. If motion tracking for speed determination is desired then the distance the structure has moved is determined at step 1804. The location information generated during motion detection may be utilized to compute distance traveled. Distance may then be referenced by time taken to travel said distance to generate structure velocity, at step 1805. Then the process proceeds to step 1806 where an inquiry is made whether to determine structure speed by alternate methods.

Otherwise, if at step 1803 motion tracking for speed determination is not desired then the process proceeds to step 1806 where an inquiry is made whether to determine structure speed by alternate methods. Alternate methods may include invasive optical readings, radioactive tagging or any alternate method as is known by those skilled in the art for speed detection. If the alternate method is desired then it may be performed at step 1807. The speed value is then output at step 1808.

Else, if at step 1806 determining structure speed by alternate methods is not desired then the process continues directly to step 1808 where speed values are output. Speed value output may include average speed values, maximum and minimum structure speed, and any additional statistical information on structure speed as is desired. The process then ends.

Pulsed echo techniques have particular implications for diagnosis of conditions such as heart murmurs and characterization of any heart sound component caused by regurgitant jet. In heart murmurs sound location in relation to specific heart valves, valve leaflet closure speed, and blood flow speeds are of particular importance for proper characterization and diagnosis of the ailment. Pulsed echo's ability to locate moving structures, such as heart valves, and determine structure speed is ideal for aiding these heart murmur diagnosis.

Additionally, pulsed echo methods may provide tissue characterization by determination of the distance of the valve to the chest wall. Said distance information may be utilized to calibrate the heart sound of that particular patient to his chest size or attenuation characteristics (the amount of subcutaneous fat, for example). Thus pulsed echo, in conjunction with attenuation information may be utilized to further provide detailed and accurate calibrations of perceived heart sounds.

### Ejection Fraction Analysis

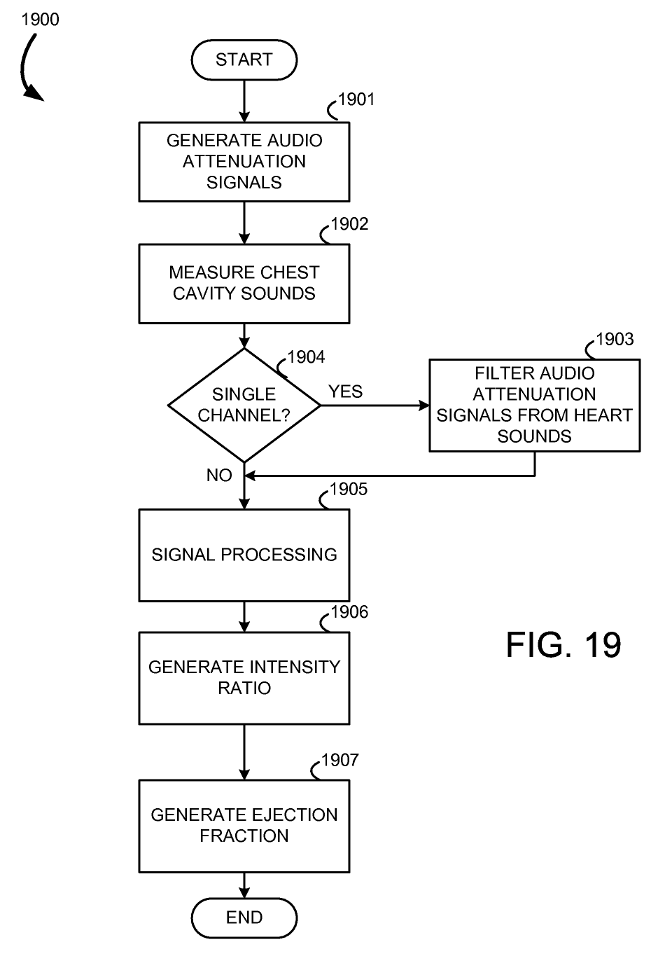

FIGS. 19 to 26 provide exemplary methodologies and examples of the utilization of the auscultatory device to determine Ejection Fraction values for heart patients. Ejection Fraction (EF) is the fraction of blood pumped out of a ventricle with each heart beat. The term ejection fraction applies to both the right and left ventricles; one can speak equally of the left ventricular ejection fraction (LVEF) and the right ventricular ejection fraction (RVEF). Without a qualifier, the term ejection fraction refers specifically to that of the left ventricle.

By definition, the volume of blood within a ventricle immediately before a contraction is known as the end-diastolic volume. Similarly, the volume of blood left in a ventricle at the end of contraction is end-systolic volume. The difference between end-diastolic and end-systolic volumes is the stroke volume, the volume of blood ejected with each beat. Ejection fraction (EF) is the fraction of the end-diastolic volume that is ejected with each beat; that is, it is stroke volume (SV) divided by end-diastolic volume (EDV). In a healthy 70-kg (154-lb) man, the SV is approximately 70 ml and the left ventricular EDV is 120 ml, giving an ejection fraction of 70/120, or 58%. Right ventricular volumes being roughly equal to those of the left ventricle, the ejection fraction of the right ventricle is normally equal to that of the left ventricle within narrow limits.

Damage to the muscle of the heart (myocardium), such as that sustained during myocardial infarction or in cardiomyopathy, impairs the heart's ability to eject blood and therefore reduces ejection fraction. This reduction in the ejection fraction can manifest itself clinically as heart failure. The ejection fraction is one of the most important predictors of prognosis; those with significantly reduced ejection fractions typically have a poorer prognoses.

FIG. 19 shows an exemplary process for using the auscultatory device to determine Ejection Fraction (EF), shown generally at step 1900. Such a process may be utilized by physicians to aid in bedside diagnostics. Additional processes may be performed utilizing the auscultatory device. The present process is intended to provide an exemplary use of the auscultatory device in a novel diagnostic technique enabled by the auscultatory device.

The process begins at step 1901 where an acoustic attenuation signal is generated from the acoustic transducer. Such an acoustic signal may be a pulse signal or a continuous acoustic signal. Additionally the acoustic signal may be at physiological frequencies or at elevated frequencies to increase resolution and eliminate interference.

The process then proceeds to step 1902 where the chest cavity of the patient is measured for sound waves. In this step a single acoustic sensor may be used to sense heart sounds and attenuation signals. In such embodiments the acoustic sensor must be able to be responsive across a wide frequency range. In some embodiments, more than one sensor may be utilized, each designed to sense acoustic signals within select frequency ranges. Moreover, at least one of the sensors, in some embodiments, may be the transducer that generates the attenuation signal. In these embodiments, the echo of the generated acoustic signal is sensed.

The process then proceeds to step 1904 where an inquiry is made if the acoustic signals are received on a single channel. If the acoustic signals are on a single channel, which is the case where a single acoustic sensor is used to sense heart sounds and attenuation signals, then the process proceeds to step 1903 where the acoustic signals are filtered by frequency. High frequency attenuation signals are thus separated from the low frequency heart sounds. The process then proceeds to step 1905, where signal processing is performed.

If at step 1904 separate channels are utilized for heart sound signals and attenuation signals then the process may proceed directly to the signal processing of step 1905.

The process then proceeds to step 1906 where intensity ratios are generated. The intensity ratio is the acoustic intensity of S1 divided by the attenuation measures Sc.

Lastly the process proceeds to step 1907 where ejection fraction may be determined. By using by the intensity ratio (S1/Sc), and the ratio between the 2 main heart sounds (S1/S2), the current ejection fraction may be estimated. The process then ends.

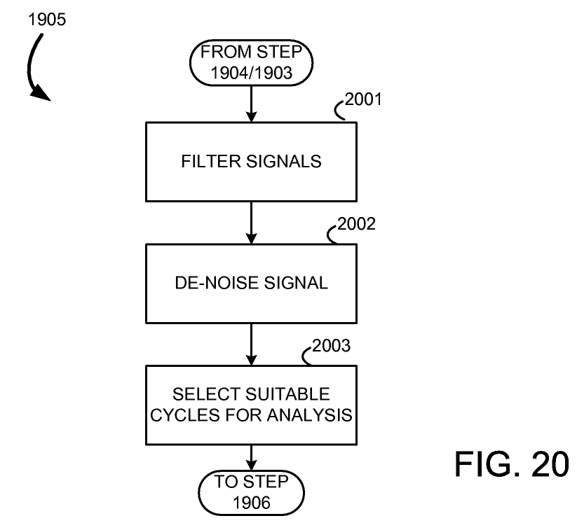

FIG. 20 shows an exemplary process for signal processing for Ejection Fraction (EF) determination, shown generally at step 1905. The process begins from step 1904 or step 1903. Then at step 2001 signals may be filtered. The process then proceeds to step 2002 where signals are de-noised. Then at step 2003 suitable cycles may be selected for analysis. In some embodiments, each patient has recordings from 3 different sites for extended durations, as well as an ECG recording. A 20 second sound recording may result in a number of heart sound cycles per site depending on the patient's heart rate. On some patients almost all of the cycles may be usable except for occasional spikes present in data. On others, there will be 2 or 3 useful cycles because of noise. In some embodiments, one method for cycle selection is to choose the median of the data. For example, all S1 and S2 amplitudes for a patient at the Pulmonic location are found. The median S1 amplitude as the representative S1 and the median S2 as the representative S2 (Note that these may not occur during the same cycle) may be selected, and then the median Signal to Noise Ratio (SNR) of all the cycles may be generated and used as the general indicator of the SNR of the entire recording. Alternate cycle selection may be utilized such as discarding all cycles below a given SNR level, use the mean of the amplitudes instead of the median, selection of the ‘best’ cycle in an entire recording (such as highest SNR) and use only the S1 and S2 from that cycle, selecting a cycle depending on a specific part of the breathing cycle, or any other appropriate cycle selection method. The process then ends by proceeding to step 1906.

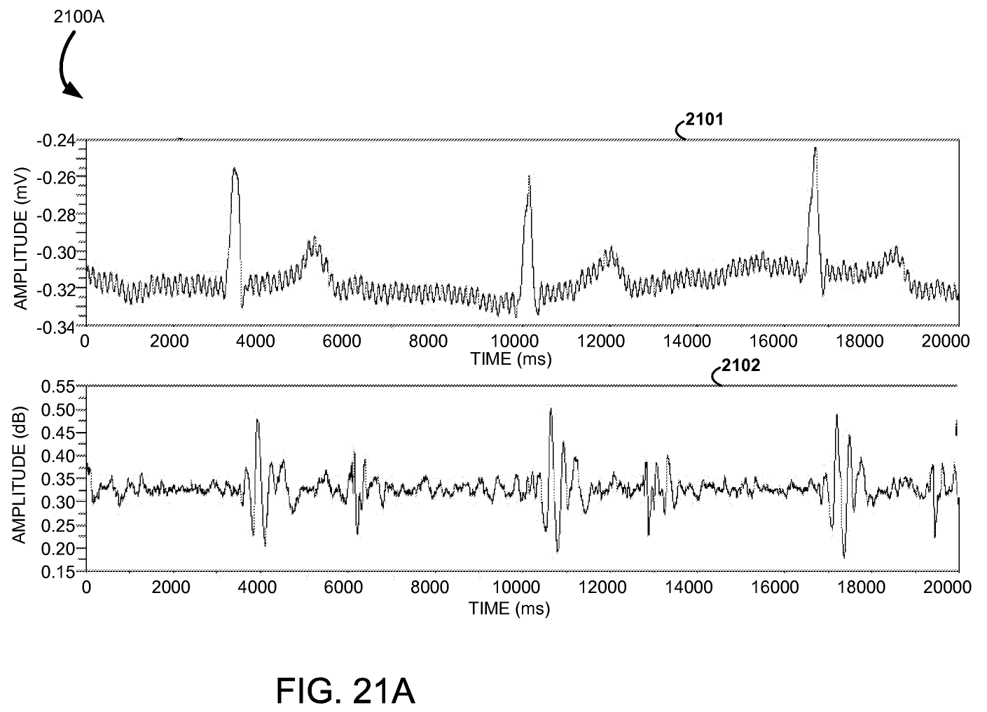

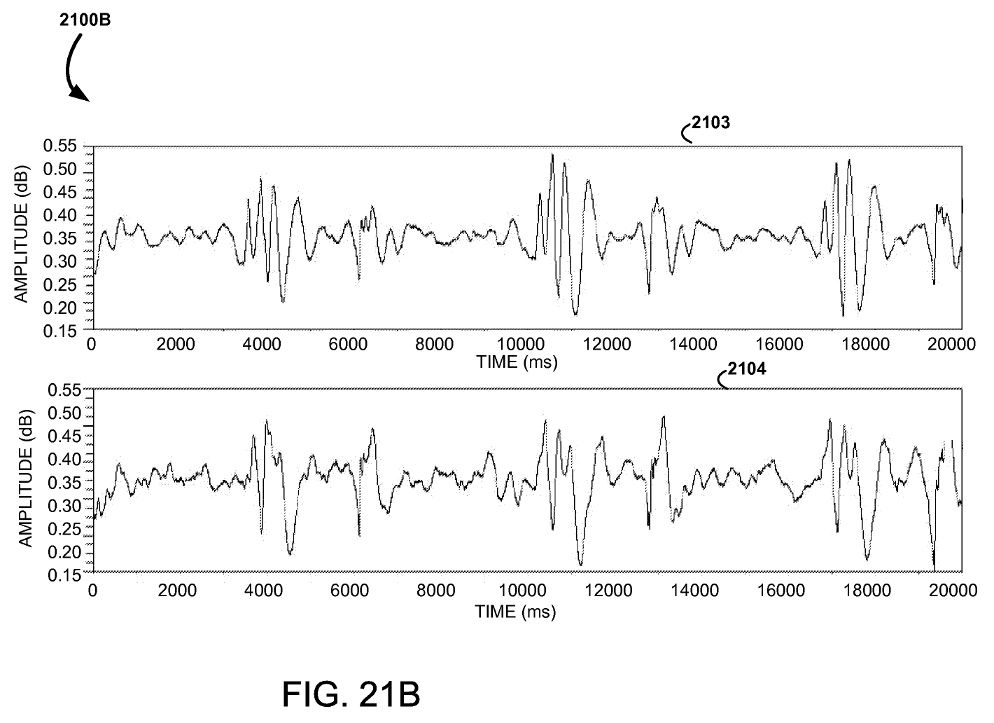

FIG. 21A shows an exemplary illustration of ECG and sound wave measurements for usage by Ejection Fraction analysis, shown generally at 2100A. The first plot 2101 is the ECG capture, and the subsequent plot is from a microphone at the Pulmonic location 2102. FIG. 21B shows an exemplary illustration of sound wave measurements for usage by Ejection Fraction analysis, shown generally at 2100B. These plots are from microphones at the Apex and Aortic locations, 2103 and 2104 respectively. Each plotting is graphed along a linear timescale. S1 is seen clearly in each plot shortly after the QRS peak in the ECG, and S2 appears shortly after the T wave.

Using the exemplary data all QRS points in the ECG data are found, which marks the beginning of each heart cycle. Since two adjacent QRS points demarcate one cycle, in the first third of that cycle, looking for the maximum and minimum signal amplitude delineates the S1 signal. The difference of maximum and minimum signal amplitude is S1 amplitude. In the next third of the cycle look once again for the maximum and minimum, the difference of which is the S2 amplitude.

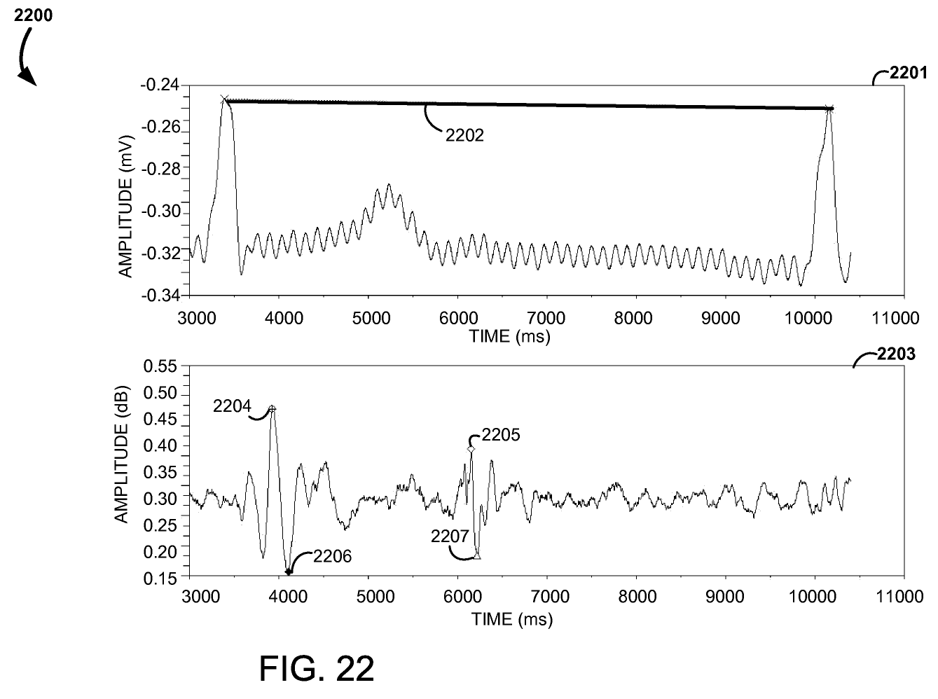

FIG. 22 shows an exemplary illustration of isolated ECG and sound wave measurement for usage by Ejection Fraction analysis, shown generally at 2200. Two Xs mark the two subsequent QRSs in the subject's ECG 2201, and there is a Line 2202 between them.

In the heart sound graph 2203, during the first third of that interval, the minimum and maximum is found 2206 and 2204 respectively. During the next third of that cycle, the minimum and maximum is found 2207 and 2205 respectively. The difference between the minima and the maxima are the amplitudes of the S1 and S2 respectively.

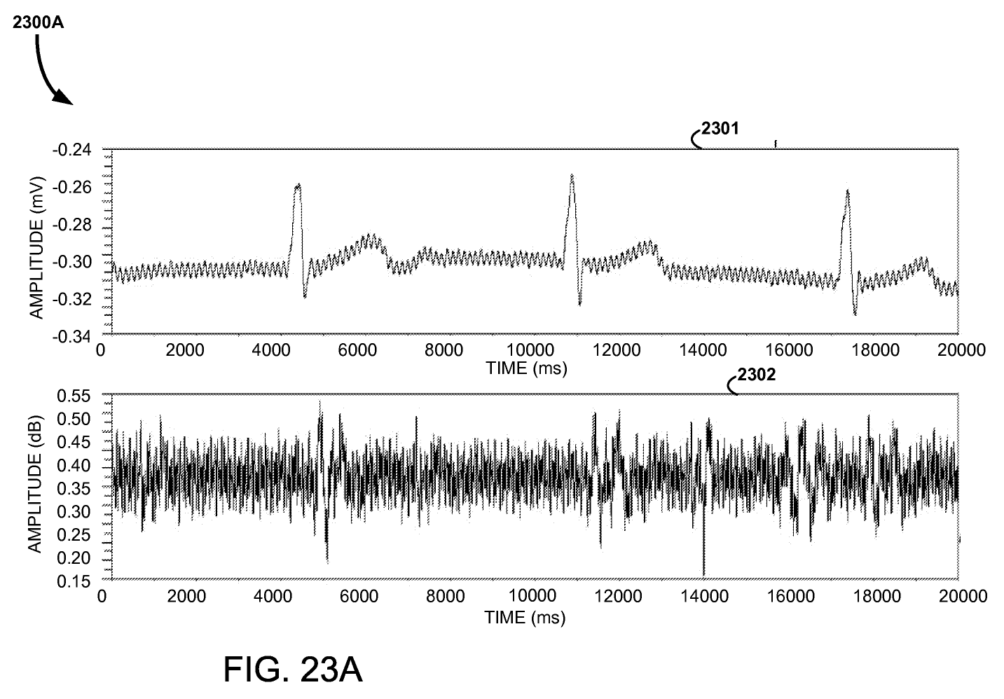

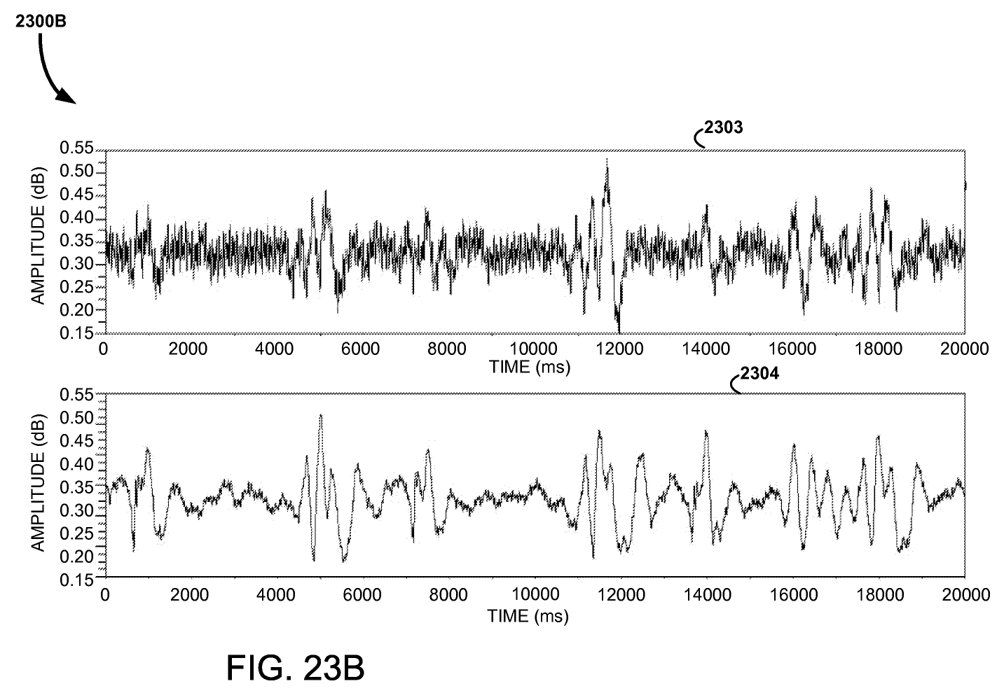

FIG. 23A shows an exemplary illustration of ECG and sound wave measurements when attenuation signal is used for Ejection Fraction analysis, shown generally at 2300A. The first plot 2301 is the ECG capture, and the subsequent plot is from the microphone at the Pulmonic 2302. FIG. 23B shows an exemplary illustration of sound wave measurements when attenuation signal is used for Ejection Fraction analysis, shown generally at 2300B. The plots are from the microphones at the Apex and Aortic locations 2303 and 2304 respectively. Each plotting is graphed along a linear timescale.

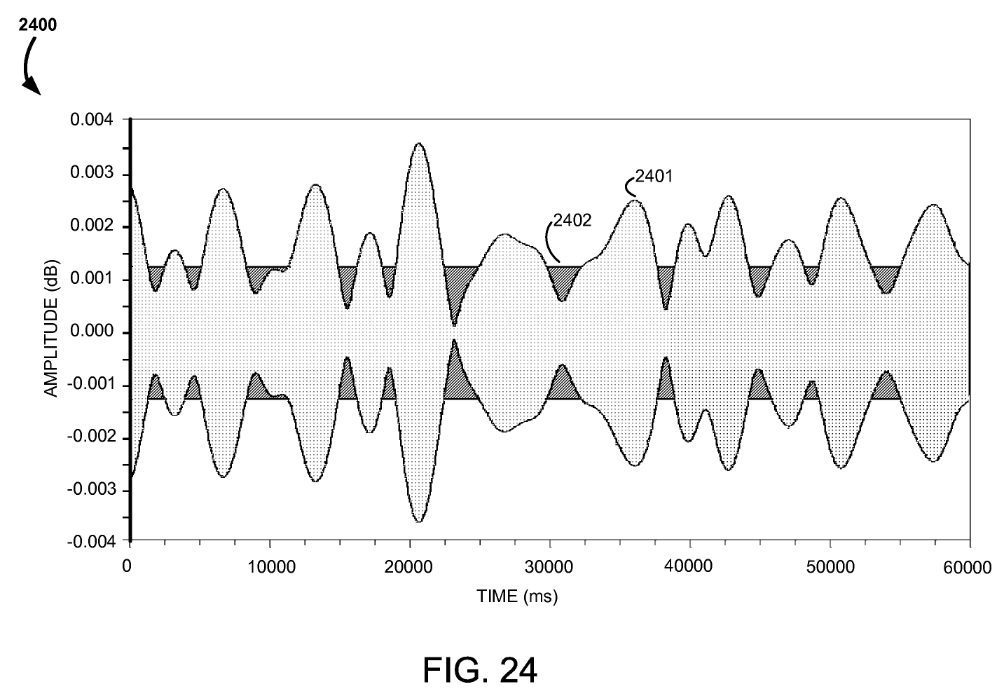

FIG. 24 shows an exemplary illustration of filtered measured transduction signals for Ejection Fraction analysis, shown generally at 2400. The data collected during the operation of the transducer is passed through a very narrow band pass filter for each tone, and the Amplitude 2401 of the output from the filter is taken as the amplitude of the tone at that location. In some embodiments, the amplitude of the tone has been defined as 4 times the Standard Deviation 2402 after the narrowband filter. Even at the narrow frequency range, the amplitude of the data fluctuates with breathing cycles and additive/subtractive effects of noise and other data within that band.

In some embodiments, the filter is a bandpass filter with appropriate cutoffs.

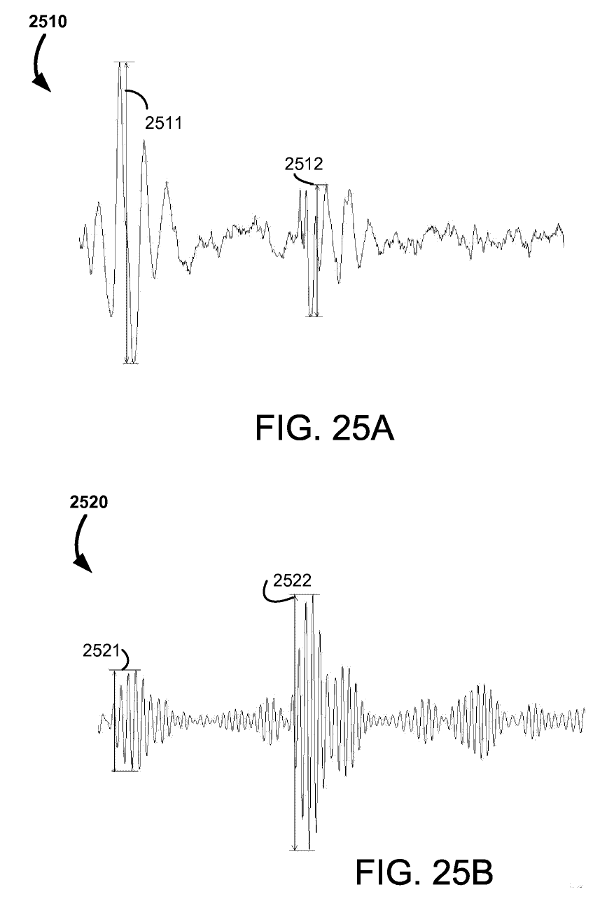

FIG. 25A shows an exemplary illustration of pre-filtered measured heart signals for Ejection Fraction analysis, shown generally at 2510. 2511 and 2512 represent the total intensity of the first and second heart sound respectively. FIG. 25B shows an exemplary illustration of post-filtered measured heart signals for Ejection Fraction analysis, shown generally at 2520. 2521 and 2522 represent the total intensity of the first and second heart sound respectively after the filtering operation.

Certain combinations of these intensities correlate well with particular pathologies. One such relationship is the ratio of the first heart sound after the filtering operation 2521 divided by the unfiltered first heart sound 2511 vs. Ejection Fraction when the filtering operation has been a band pass operation centered around a particular frequency band. Another such relationship has been the ratio of the unfiltered first heart sound 2511 divided by the unfiltered second heart sound 2512 vs. Ejection Fraction.

It is quite conceivable that there are other relationships between the mentioned intensities 2511, 2512, 2521, 2522 and other cardiac measures. Some of these ratios may also correlate well with the presence of certain cardiac pathologies. For example, after a particular filtering operation, the value of a particular ratio such as the filtering operation 2521 divided by the unfiltered first heart sound 2511 may indicate the presence of a particular cardiac disease such as Aortic Stenosis or Mitral Regurgitation.

Some of these relationships may involve looking at multiple ratios after multiple filtering operations, and a particular pathology might have a distinct frequency signature, whereby looking at a number of ratios over a number of filtering operations might indicate the subject's pathology.

The filtering operations may involve simple filters, for example a straightforward analog nth order bandpass/lowpass/highpass filter. It is conceivable that wavelet operations, which by their nature divide up the signal into various frequency bands, can also be used to carry out measurements on the heart sound signal.

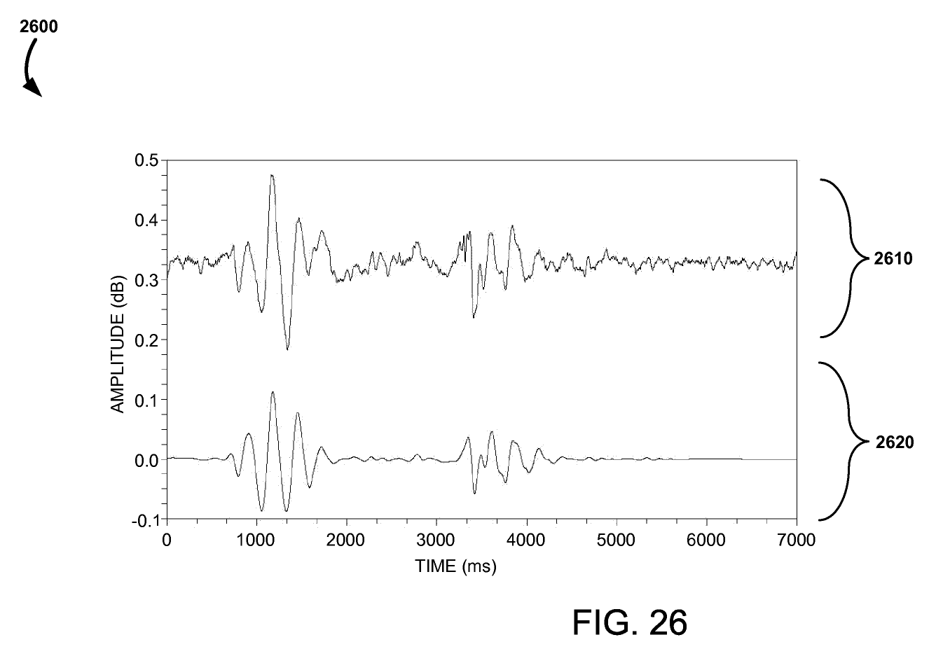

FIG. 26 shows an exemplary illustration of measured heart signals before and after de-noising for Ejection Fraction analysis, shown generally at 2600. Issues of noisy recording may affect the intensity of the heart sound parameters being analyzed. This issue may, in some embodiments, be dealt with on two separate levels. First of all, by developing a measure for the noise level within the signal a threshold may be developed to decide whether a particular heart cycle is clean enough to include in measurements. Secondly, data may be cleansed via de-noising.

Signal to Noise Ratio (SNR) may be determined and utilized in the de-noising process. Measuring the noise level, at least in the context of heart sound study, is to measure the power of the signal in a ‘good’ region compared to the power of the signal in a ‘noise’ region.

In some embodiments, the entire signal is filtered with a bandpass filter in the frequency range of the first and second heart sounds.

Wavelet de-noising works quite effectively in removing Gaussian type noise. The de-noising is done on a cycle by cycle basis (as opposed to de-noising the entire capture in one go). This does not have a huge impact, except that the noise cutoff thresholds are chosen on a cycle by cycle basis as opposed to one noise threshold for the entire cycle. The graph illustrated at 2610 is an example of recorded heart sounds before de-noising. The graph illustrated at 2620 is an example of the same recorded heart sounds after de-noising.

One novel aspect of the present invention is that all of the capabilities described may be performed in the background—i.e. the image processing extraction of the valve from the motion trace, distance measurements, signal processing for speed determination, Doppler frequency shift, and blood flow estimation. The physicians, or other users, require no new skills to effectuate the system.

Additionally, display of information may be defined based upon statistical confidence levels to minimize misdiagnosis and provide user recommendations. For example, if the valve responsible for a murmur is not reliably detected, say, over 50% of the cardiac cycle, the sensor/transducer may not be pointing in a stable fashion due to hand motion etc, it may indicate repositioning or provide feedback to the user and likewise indicate when the sensor/transducer is pointing accurately at a valve or provide feedback to maximize the motion trace indicating a look direction that sees maximum travel of the leaflet.

Modifications of the present invention are also possible. For example, it is possible to incorporate noise cancellation capability to the embodiments described above, thereby substantially removing ambient environmental noises from the heart sounds received by the acoustic sensors, e.g., sensors of chest-pieces 500, 600, 700.

In sum, the present invention provides many advantages over the existing auscultatory devices, including ease of use, improved accuracy, portability, and cost effectiveness. The present invention also allows for the concurrent gathering of acoustic and electrical heart information from the patient by combining an acoustic sensing with an ECG sensing.

While a number of preferred embodiments have been illustrated as applying to the measurement and calibration for heart sounds, the present invention is intended for the measurement of any suitable sounds, including but not limited to lung sounds, fetal sounds, and applications on sound processing on inorganic medium.

While this invention has been described in terms of several preferred embodiments, there are alterations, modifications, permutations, and substitute equivalents, which fall within the scope of this invention. Although sub-section titles have been provided to aid in the description of the invention, these titles are merely illustrative and are not intended to limit the scope of the present invention.

It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A method for measuring acoustic attenuation in a subject, the method comprising:
generating a first acoustic signal at a first location of the subject;
receiving an attenuated acoustic signal resulting from the first acoustic signal, wherein the attenuated acoustic signal is received at a second location of the subject; and
computing an acoustic attenuation between the first location and the second location based on differences between the first acoustic signal and the attenuated acoustic signal.

2. The method of claim 1 wherein the first location is closely located to the second location.

3. The method of claim 1 wherein the first location and the second location are located on a thoracic region of the subject.

4. The method of claim 1 wherein the second location is a standard ECG position.

5. The method of claim 1 further comprising:
sensing at least one heart sound at the second location of the subject; and
normalizing the at least one heart sound based on the computed acoustic attenuation.

6. An auscultation system useful in association with a subject, the system comprising:
a transducer configured to generate a first acoustic signal at a first location of the subject;
a sensor configured to receive an attenuated acoustic signal resulting from the first acoustic signal, wherein the attenuated acoustic signal is received at a second location of the subject; and
a signal processor configured to compute an acoustic attenuation between the first location and the second location based on differences between the first acoustic signal and the attenuated acoustic signal.

7. The system of claim 6 wherein the first location is closely located to the second location.

8. The system of claim 6 wherein the first location is located at the same location as the second location.

9. The system of claim 8 wherein the transducer includes the sensor.

10. The system of claim 6 wherein the first location and the second location are located on a thoracic region of the subject.

11. The system of claim 6 wherein the second location is a standard ECG position.

12. The system of claim 6 wherein the sensor is further configured to sense at least one heart sound at the second location of the subject, and wherein the signal processor is further configured to normalize the at least one heart sound based on the computed acoustic attenuation.

13. The system of claim 6 further comprising a noise canceller.

14. A method for calibrating heart sounds of a subject, useful in association with an auscultation device having a transducer, a sensor and a heart sound processor, the method comprising:
orienting the transducer on a first location of the subject;
orienting the sensor on a second location of the subject;
generating an audio signal at the first location of the subject by utilizing the transducer;
receiving an attenuated audio signal resulting from the generated audio signal, and wherein the attenuated audio signal is received at the second location of the subject by the sensor;
receiving a heart sound signal at the second location of the subject by utilizing the sensor;
computing an acoustic attenuation between the first location and the second location based on differences between the generated audio signal and the received attenuated audio signal; and
calibrating the heart sound signal by utilizing the computed acoustic attenuation.

15. The method of claim 14 further comprising:
filtering the attenuated audio signal from the heart sound signal; and
conditioning the heart sound signal.

16. The method of claim 14 wherein the first position and the second position are located in a substantially close proximity.

17. A method for pulse echo auscultatory diagnosis of a subject, useful in association with an auscultation device having an echo transducer and a heart sound processor, the method comprising:
orienting the echo transducer on the subject;
generating a first audio signal pulse from the echo transducer;
receiving a first return echo of the audio signal pulse, wherein the first return echo is received by the echo transducer;
generating a first brightness encoded image from the first received return echo, wherein the first brightness encoded image represents internal structures of the subject, and wherein the first received return echo provides location data on the internal structures of the subject;
receiving a heart sound signal of the subject; and
calibrating the heart sound signal by utilizing the first brightness encoded image, wherein calibrating the heart sound signal includes relating acoustic properties of tissues to the represented internal structures of the subject.

18. The method of claim 17 further comprising:
filtering the first audio signal pulse from the heart sound signal; and
conditioning the heart sound signal.

19. The method of claim 17 further comprising:
generating a second audio signal pulse from the echo transducer, wherein the first and second audio signal are interleaved in relation to subject's cardiac cycle;
receiving a second return echo of the audio signal pulse, wherein the second return echo is received by the echo transducer;
generating a second brightness encoded image from the second received return echo, wherein the second brightness encoded image represents internal structures of the subject, and wherein the second received return echo provides location data on the internal structures of the subject; and
detecting motion of the internal structures of the subject by comparing the first brightness encoded image and the second brightness encoded image for discrepancies.

20. The method of claim 17 further comprising:
detecting distance of the moving internal structure of the subject by comparing the first brightness encoded image and the second brightness encoded image; and
computing speed of the moving internal structure by referencing the distance traveled by a time differential, wherein the time differential is computed by comparing times of generation of the first acoustic pulse and the second acoustic pulse.

21. The method of claim 19 further comprising determining speed of the moving internal structure by detecting Doppler shift between the first generated acoustic pulse and the first received echo.

22. The method of claim 17 further comprising generating operating suggestions, wherein the operating suggestions are generated by statistical analysis of brightness encoded image.

23. A method for measuring ejection fraction of a subject, useful in association with an auscultation device having a transducer, a sensor and a heart sound processor, the method comprising:
orienting the transducer on a first location of the subject;
orienting the sensor on a second location of the subject;
generating an audio signal at the first location of the subject by utilizing the transducer;
receiving an attenuated audio signal resulting from the generated audio signal, wherein the attenuated audio signal is received at the second location of the subject by the sensor;
receiving a heart sound signal at the second location of the subject by the sensor;
computing an acoustic attenuation between the first location and the second location based on differences between the generated audio signal and the received attenuated audio signal;
computing an intensity ratio by dividing an amplitude of the conditioned heart sound signal by the acoustic attenuation; and
computing ejection fraction of the heart subject by correlation to the computed intensity ratio.

24. The method of claim 23 further comprising:
filtering the attenuated audio signal from the heart sound signal; and
conditioning the heart sound signal.
